(19) United States
(12) Patent Application Publication
(10) Pub. No.: US 2020/0102356 A1
(43) Pub. Date: Apr. 2, 2020

(54) Compositions and Methods for the Treatment of Huanglongbing (HLB) aka Citrus Greening in Citrus Plants

(71) Applicant: Innate Immunity, LLC, Santa Fe, NM (US)

(72) Inventors: Goutam Gupta, Los Alamos, NM (US); Ed Stover, Fort Pierce, FL (US)

(73) Assignee: NMC Inc., Los Alamos, NM (US)

(21) Appl. No.: 16/148,848

(22) Filed: Oct. 1, 2018

(51) Int. Cl. C07K 14/415; C12N 15/82

(52) U.S. Cl. CPC C07K 14/415; C12N 15/8239; C12N 15/8281

(57) ABSTRACT

The invention may include engineered antimicrobial peptides to treat HLB disease, preferably in citrus plants. Specifically, the invention may include novel antimicrobial peptide derived from amphipathic helical peptides that may further be used to treat HLB disease in citrus plants. In one embodiment, the invention may include an engineered antimicrobial peptide formed by coupling two amphipathic helical peptides. Specifically, a generalized antimicrobial peptide of the invitation may include a first amphipathic helical peptide coupled with a second amphipathic helical peptide by a linker domain forming a helix-turn-helix scaffold formation. Such amphipathic helical peptides may be endogenous to a target host, preferably a citrus plant.

### GOVERNMENT INTEREST

This invention was made with government support under National Institute of Food and Agriculture (NIFA), USDA, Citrus Greening award #2015-70016-23028-S15191.

### TECHNICAL FIELD

The inventive technology includes novel systems, methods, and compositions for the treatment of bacterial infections in citrus plants. The invention may specifically include novel systems, methods, and compositions for the treatment and prevention of Huanglongbing in citrus plants. Further embodiments include novel engineered antimicrobial peptide compositions and their use.

### BACKGROUND

Huanglongbing (HLB) a/k/a/Citrus Greening (CG) disease is a vector-borne disease, caused by the transmission of gram-negative Candidatus Liberibacter by insect psyllids. Asian citrus psyllid (ACP) and Candidatus Liberibacte asiaticus (CLas) are respectively the transmitting vectors and causative organism of HLB in the US. Other than tree removal, there is no effective control once a tree is infected and there is no known cure for the disease. Infected trees may produce misshapen, unmarketable, and bitter fruit. HLB reduces the quantity and quality of citrus fruits, eventually rendering infected trees useless. In areas of world affected by HLB the average productive lifespan of citrus trees has dropped from 50 or more years to 15 or less. The trees in the orchards usually die 3-5 years after becoming infected and require removal and replanting.

Citrus plants infected by the HLB bacteria may not show symptoms for years following infection. Initial symptoms frequently include the appearance of yellow shoots on a tree. As the bacteria move within the tree, the entire canopy progressively develops a yellow color. The most characteristic symptoms of HLB are a blotchy leaf mottle and vein yellowing that develops on leaves attached to shoots showing the overall yellow appearance.

HLB disease has devastated the Florida citrus industry since the disease was first encountered approximately seven years ago. Although it is not yet widespread in Texas and California, HLB is looming large on these two citrus producing states. As noted above, with no known cure, efforts have been placed on preventing the spread of HLB. As with all vector-borne diseases, insecticides were tried first to stop the spread of the HLB. However, in Florida the number of Liberibacter-carrying psyllids is too many and too overwhelming for psyllid control by insecticides. In fact, over eighty percent of the Florida citrus trees are already currently infected. In states like Texas and California, psyllid control is still being tried with limited success. However, increasing disease pressure may soon render psyllid control ineffective.

Another method for ameliorating the effects in HLB infected citrus includes the direct application of antibiotic compounds. Currently, antibiotic streptomycin is sprayed to reduce the Liberibacter load from the infected citrus plants. However, the use of streptomycin poses several drawbacks, namely: (i) poor activity in Liberibacter clearance; (ii) potentially being toxic to citrus and human; and (iii) generation of Liberibacter resistance in citrus, which may be transferred to human. Thus, there exists a need for an effective solution to protect the $50B dollars US citrus industry from HLB.

The inventive technology overcomes the limitations outlined above. In particular, the inventive technology includes a novel HLB treatment of infected citrus. In one preferred embodiment, the inventive technology includes a new class of engineered peptides that are superior to the use of antibiotics in that they are devoid of the drawbacks outlined above. The inventive technology specifically includes the use of modified host amphipathic antimicrobial peptides (AAPs), which are present in insects, plants, mammals, and humans and act as an important part of innate immune repertoire. These AAPs can kill the invading pathogens as well as induce host innate immune defense upon pathogen attack. It may be noted that these short AAPs are present in insects, mammals, and humans as independent molecules whereas in plants these are present as a part of other proteins. However, these plant peptides when synthesized and treated on pathogens (particularly gram-negative bacteria) show antimicrobial activity. After their discovery about three decades ago, these amphipathic antimicrobial peptides raised a lot of hope as a superior alternative to antibiotics due to the following reasons:

First, while traditional antibiotics target DNA, RNA, protein, and/or cell wall synthesis machineries inside the bacteria, AAPs target the bacterial membrane from the outside. Therefore, they may be effective on antibiotic resistant bacteria. Second, AAPs may be derived from the host such that they may be reasonably non-toxic. Third, AAPs are easy to synthesize. And finally, AAPs are considered drugs (and not biologics) and therefore, they are not under strict regulatory and other legal constraints.

Despite these advantages, there exist several important drawbacks to the use of traditional AAPs as anti-microbial agents. For example, traditional AAPs have shown bactericidal activity only at high concentrations at which they may be toxic to the host. In addition, bacteria develop resistance against them by modifying their outer membranes. Note that specific membrane modifications hinder the three key steps in the action of AAPs, namely attachment, insertion, and rupture of the bacterial membrane.

Others have tried to use such traditional AAPs as anti-microbial agents with limited success. For example, some have proposed the therapeutic use of traditional AAPs to target certain bacterial infection. There are other examples of transgenic plants expressing helical amphipathic antibacterial peptides derived from plant or non-plant hosts (See e.g., U.S. Pat. Nos. 6,235,973, 8,906,365, PCT Application No. PCT/US2008/070612, U.S. Pat. Nos. 5,861,478, 9,807,720, and 9,522,942, each reference being incorporated herein in their entireties). One way or another, such traditional systems and techniques have failed to address the limitations outlined above. As such, there further exists a need for more effective AAPs that may be used as antimicrobial agents.

As described generally below, the inventive technology includes novel modified AAPs based upon endogenous plant peptides such that are more efficient in causing attachment, insertion, and rupture of the bacterial membrane and that are non-toxic to the host cell. Neither are the invention's modified AAPs susceptible to bacterial resistances since they can overcome the barriers in attachment, insertion, and rupture of the bacterial membrane posed by bacterial resistance.

### SUMMARY OF THE INVENTION

On aspect of the current inventive technology includes novel systems, methods, and compositions for the treatment of HLB disease, preferably in citrus plants. One general aspect of the invention may include novel antimicrobial peptides having a helix-turn-helix scaffold formation that exhibit: i) increased bactericidal effects; 2) increased efficiency of attachment and/or insertion into a bacterial membrane; and iii) a lower susceptibility to bacterial resistance. In one preferred aspect, such novel helix-turn-helix scaffold antimicrobial peptides may be used as a therapeutic composition for the treatment of bacterial infections. In a preferred aspect, such novel helix-turn-helix scaffold antimicrobial peptides may be used as a therapeutic composition for the treatment of gram-negative bacterial infections in plants. Finally, in another preferred aspect, such novel helix-turn-helix scaffold antimicrobial peptides may be used as a therapeutic composition for the treatment of CLas, a causative agent of HLB disease in citrus plants.

One aspect of the inventive technology may include a novel a antimicrobial peptide comprising a first amphipathic helical peptide and a second amphipathic helical peptide coupled by a linker domain forming a helix-turn-helix scaffold formation wherein said helix-turn-helix scaffold formation has: 1) increased bactericidal effects compared to a single endogenous amphipathic helical peptide; 2) increased efficiency of attachment and/or insertion into a bacterial membrane compared to a single endogenous amphipathic helical peptide; 3) lower susceptibility to bacterial resistance compared to a single endogenous amphipathic helical peptide; and 4) low or no toxicity to mammalian cells; and 5) low or no phytotoxcicity to plant cells.

One aspect of the current inventive technology may include a novel antimicrobial peptide having a first amphipathic helical peptide and a second amphipathic helical peptide coupled by a linker domain forming a helix-turn-helix scaffold formation.

Additional aspects of the inventive technology may include embodiments wherein a first amphipathic helical peptide and a second amphipathic helical peptide are both endogenous amphipathic helical peptides from a citrus plant.

Additional aspects of the inventive technology may include embodiments wherein a first amphipathic helical peptide and/or a second amphipathic helical peptide are each selected from the group consisting of: P11, 11P1, 12P, 12P1, 12P-2, 10P, and 27P, or any combination thereof.

Additional aspects of the inventive technology may include embodiments wherein a first amphipathic helical peptide and/or a second amphipathic helical peptide are each selected from the group consisting of: SEQ ID NOs. 1-2, 13-15, 19, and 21, or any combination thereof.

Additional aspects of the inventive technology may include embodiments wherein the linker domain comprises a peptide linker having at least four amino acids.

Additional aspects of the inventive technology may include embodiments wherein the linker domain comprises a GPGR-turn having an amino acid sequence identified as SEQ ID NO. 23.

Additional aspects of the inventive technology may include embodiments wherein a first amphipathic helical peptide and a second amphipathic helical peptide are the same amphipathic helical peptide.

Additional aspects of the inventive technology may include embodiments wherein the antimicrobial peptide is selected from the group consisting of: P26, 26P1, 26P2, 26P3, 26P4, 26P5, cysP30, 41P, 28P, 28P1, 28P1-2, 24P, and 58-P.

Additional aspects of the inventive technology may include embodiments wherein the antimicrobial peptide is selected from the group consisting of: SEQ ID NOs. 3-12, 16-18, 20, and 22-23, or a variant thereof.

Additional aspects of the inventive technology may include embodiments wherein an antimicrobial peptide is encoded by a polynucleotide comprising a nucleic acid sequence.

Additional aspects of the inventive technology may include embodiments wherein the antimicrobial peptide is encoded by a polynucleotide which is further linked to a promoter to produce an expression vector.

Additional aspects of the inventive technology may include embodiments wherein the antimicrobial peptide is encoded by a polynucleotide operably linked to a promotor, and wherein a plant or plant cell produce the antimicrobial peptide. In a preferred aspect, such a plant or plant cell may include a citrus plant or citrus plant cell.

Additional aspects of the inventive technology may include embodiments wherein for the antimicrobial peptide may be used as a therapeutic agent for plants infected with and/or at risk of being infected by a bacterial pathogen.

Additional aspects of the inventive technology may include embodiments wherein the antimicrobial peptide may be used as a therapeutic agent for plants infected with and/or at risk of being infected by Candidatus Liberibacte asiaticus (CLas).

Additional aspects of the inventive technology may include embodiments wherein the antimicrobial peptide may be topically applied to plants infected with and/or at risk of being infected by CLas.

Additional aspects of the inventive technology may include embodiments wherein the antimicrobial peptide may be used as a therapeutic agent for the treatment and/or prevention of Huanglongbing (HLB).

Additional aspects of the inventive technology may include embodiments wherein at least one hydrophobic amino acid residue from each of the amphipathic helical peptides are replaced with a cysteine residue forming a disulfide bridge between the amphipathic helical peptides.

Additional aspects of the inventive technology may include embodiments wherein a first amphipathic helical peptide and a second amphipathic helical peptide coupled by a linker domain forming a helix-turn-helix scaffold formation has increased bactericidal effects compared to a single endogenous amphipathic helical peptide.

Additional aspects of the inventive technology may include embodiments wherein a first amphipathic helical peptide and a second amphipathic helical peptide coupled by a linker domain forming a helix-turn-helix scaffold formation having increased efficiency of attachment and/or insertion into a bacterial membrane compared to a single endogenous amphipathic helical peptide. In a preferred embodiment, a bacterial membrane may be a gram-negative bacterial membrane.

Additional aspects of the inventive technology may include embodiments wherein a first amphipathic helical peptide and a second amphipathic helical peptide coupled by a linker domain forming a helix-turn-helix scaffold formation has a lower susceptibility to bacterial resistance compared to a single endogenous amphipathic helical peptide.

Another aspect of the current inventive technology may include a novel antimicrobial peptide having two P11 amphipathic helical peptides coupled by a linker domain forming a helix-turn-helix scaffold formation identified as amino acid SEQ ID NO. 3.

Additional aspects of the inventive technology may include embodiments wherein the P11 amphipathic helical peptides are both endogenous P11 amphipathic helical peptides from a citrus plant.

Additional aspects of the inventive technology may include embodiments wherein the linker domain comprises a peptide linker having at least four amino acids.

Additional aspects of the inventive technology may include embodiments wherein the linker domain comprises a GPGR-turn having an amino acid sequence identified as SEQ ID NO. 23.

Additional aspects of the inventive technology may include embodiments wherein at least one hydrophobic amino acid residue from each of the P11 amphipathic helical peptides are replaced with a cysteine residue forming a disulfide bridge between the P11 amphipathic helical peptides.

Additional aspects of the inventive technology may include embodiments wherein at least one hydrophobic amino acid residue from each of the P11 amphipathic helical peptides are replaced with a cysteine residue forming a disulfide bridge between the P11 amphipathic helical peptides and may further be identified as amino acid SEQ ID NO. 9.

Additional aspects of the inventive technology may include embodiments wherein a second linker domain may be coupling the two P11 amphipathic helical peptides forming a cyclic scaffold formation identified as amino acid SEQ ID NO. 11.

Additional aspects of the inventive technology may include embodiments wherein the antimicrobial peptide is encoded by a polynucleotide comprising a nucleic acid sequence.

Additional aspects of the inventive technology may include embodiments wherein the antimicrobial peptide is encoded by a polynucleotide and linked to a promoter to produce an expression vector.

Additional aspects of the inventive technology may include embodiments wherein a genetically altered plant or plant cell comprising the above polynucleotide may be configured to produce an antimicrobial peptide and is operably linked to a promotor, wherein the plant or plant cell may produce the antimicrobial peptide. In a preferred aspect, such a plant or plant cell may include a citrus plant or citrus plant cell.

Additional aspects of the inventive technology may include embodiments wherein for use as a therapeutic agent for plants infected with and/or at risk of being infected by a bacterial pathogen.

Additional aspects of the inventive technology may include embodiments wherein the antimicrobial peptide may be used as a therapeutic agent for plants infected with and/or at risk of being infected by Candidatus Liberibacte asiaticus (CLas).

Additional aspects of the inventive technology may include embodiments wherein the antimicrobial peptide may be topically applied to plants infected with and/or at risk of being infected by CLas.

Additional aspects of the inventive technology may include embodiments wherein the antimicrobial peptide may be used as a therapeutic agent for the treatment and/or prevention of Huanglongbing (HLB).

Additional aspects of the inventive technology may include embodiments wherein at least one hydrophobic amino acid residue from each of the amphipathic helical peptides are replaced with a cysteine residue forming a disulfide bridge between the amphipathic helical peptides.

Additional aspects of the inventive technology may include embodiments wherein a first amphipathic helical peptide and a second amphipathic helical peptide coupled by a linker domain forming a helix-turn-helix scaffold formation has increased bactericidal effects compared to a single endogenous amphipathic helical peptide.

Additional aspects of the inventive technology may include embodiments wherein a first amphipathic helical peptide and a second amphipathic helical peptide coupled by a linker domain forming a helix-turn-helix scaffold formation having increased efficiency of attachment and/or insertion into a bacterial membrane compared to a single endogenous amphipathic helical peptide. In a preferred embodiment, a bacterial membrane may be a gram-negative bacterial membrane.

Additional aspects of the inventive technology may include embodiments wherein a first amphipathic helical peptide and a second amphipathic helical peptide coupled by a linker domain forming a helix-turn-helix scaffold formation has a lower susceptibility to bacterial resistance compared to a single endogenous amphipathic helical peptide.

Another aspect of the current inventive technology may include a novel antimicrobial peptide comprising two amphipathic helical peptides coupled by a linker domain forming a helix-turn-helix scaffold formation and wherein at least one hydrophobic amino acid residue from each of the amphipathic helical peptides are replaced with a cysteine residue forming a disulfide bridge between the amphipathic helical peptides.

Additional aspects of the inventive technology may include embodiments wherein the antimicrobial peptide of claim 39 wherein the first amphipathic helical peptide and the second amphipathic helical peptide are both endogenous amphipathic helical peptides from a citrus plant.

Additional aspects of the inventive technology may include the antimicrobial peptide described above wherein a first amphipathic helical peptide and a second amphipathic helical peptide are each selected from the group consisting of: P11, 11P1, 12P, 12P1, 12P-2, 10P, and 27P, or any combination thereof.

Additional aspects of the inventive technology may include the antimicrobial peptide of described above wherein a first amphipathic helical peptide and a second amphipathic helical peptide are each selected from the group consisting of: SEQ ID NO. 1-2, 13-15, 19, and 21, or any combination thereof.

Additional aspects of the inventive technology may include the antimicrobial peptide described above wherein the linker domain comprises a peptide linker having at least four amino acids.

Additional aspects of the inventive technology may include the antimicrobial peptide described above wherein the linker domain comprises a GPGR-turn having an amino acid sequence identified as SEQ ID NO. 23.

Additional aspects of the inventive technology may include the antimicrobial peptide described above wherein a first amphipathic helical peptide and a second amphipathic helical peptide are the same amphipathic helical peptide.

Additional aspects of the inventive technology may include the antimicrobial peptide described above wherein the antimicrobial peptide is identified as amino acid SEQ ID NO. 9.

Additional aspects of the inventive technology may include the antimicrobial peptide described above which is encoded by a polynucleotide comprising a nucleic acid sequence.

Additional aspects of the inventive technology may include embodiments wherein the polynucleotide described above is linked to a promoter to produce an expression vector.

Additional aspects of the inventive technology may include embodiments wherein a genetically altered plant or plant cell comprising the polynucleotide described above is operably linked to a promotor, and wherein the plant or plant cell produce the antimicrobial peptide. In a preferred aspect, such a plant or plant cell may include a citrus plant or citrus plant cell.

Additional aspects of the inventive technology may include embodiments wherein the antimicrobial peptide described above may be used as a therapeutic agent for plants infected with and/or at risk of being infected by a bacterial pathogen.

Additional aspects of the inventive technology may include embodiments wherein the antimicrobial peptide described above may be used as a therapeutic agent for plants infected with and/or at risk of being infected by Candidatus Liberibacte asiaticus (CLas).

Additional aspects of the inventive technology may include embodiments wherein the composition or antimicrobial peptide described above may be topically applied to plants infected with and/or at risk of being infected by CLas.

Additional aspects of the inventive technology may include embodiments wherein the composition described above may be used as a therapeutic agent for the treatment and/or prevention of Huanglongbing (HLB).

Additional aspects of the inventive technology may include embodiments wherein the antimicrobial peptide described above has two amphipathic helical peptides coupled by a linker domain forming a helix-turn-helix scaffold formation and wherein at least one hydrophobic amino acid residue from each of the amphipathic helical peptides are replaced with a cysteine residue forming a disulfide bridge between the amphipathic helical peptides and has increased bactericidal effects compared to a single endogenous amphipathic helical peptide.

Additional aspects of the inventive technology may include embodiments wherein the antimicrobial peptide described above has two amphipathic helical peptides coupled by a linker domain forming a helix-turn-helix scaffold formation and wherein at least one hydrophobic amino acid residue from each of the amphipathic helical peptides are replaced with a cysteine residue forming a disulfide bridge between the amphipathic helical peptides and has increased efficiency of attachment and/or insertion into a bacterial membrane compared to a single endogenous amphipathic helical peptide.

Additional aspects of the inventive technology may include embodiments wherein the antimicrobial peptide described above has two amphipathic helical peptides coupled by a linker domain forming a helix-turn-helix scaffold formation and wherein at least one hydrophobic amino acid residue from each of the amphipathic helical peptides are replaced with a cysteine residue forming a disulfide bridge between the amphipathic helical peptides has a lower susceptibility to bacterial resistance compared to a single endogenous amphipathic helical peptide.

Additional aspects of the inventive technology may include embodiments wherein the antimicrobial peptide described above may further comprise a second linker domain coupling the two P11 amphipathic helical peptides forming a cyclic scaffold formation identified as amino acid SEQ ID NO. 11.

Another aspect of the current inventive technology may include a novel antimicrobial peptide comprising two P11 amphipathic helical peptides coupled by a linker domain forming a helix-turn-helix scaffold formation and wherein at least one hydrophobic amino acid residue from each of the P11 amphipathic helical peptides are replaced with a cysteine residue forming a disulfide bridge between the P11 amphipathic helical peptides identified as amino acid SEQ ID NO. 9.

Additional aspects of the inventive technology may include the antimicrobial peptide described above wherein the P11 amphipathic helical peptides are both endogenous P11 amphipathic helical peptides from a citrus plant.

Additional aspects of the inventive technology may include the antimicrobial peptide described above wherein the linker domain comprises a peptide linker having at least four amino acids.

Additional aspects of the inventive technology may include the antimicrobial peptide described above wherein the linker domain comprises a GPGR-turn having an amino acid sequence identified as SEQ ID NO. 23.

Additional aspects of the inventive technology may include the antimicrobial peptide described above encoded by a polynucleotide comprising a nucleic acid sequence.

Additional aspects of the inventive technology may include embodiments wherein the polynucleotide described above is linked to a promoter to produce an expression vector.

Additional aspects of the inventive technology may include a genetically altered plant or plant cell comprising the polynucleotide described above operably linked to a promotor, wherein the plant or plant cell produce the antimicrobial peptide. In a preferred aspect, such a plant or plant cell may include a citrus plant or citrus plant cell.

Additional aspects of the inventive technology may include embodiments wherein the antimicrobial peptide described above may be used as a therapeutic agent for plants infected with and/or at risk of being infected by a bacterial pathogen.

Additional aspects of the inventive technology may include embodiments wherein the antimicrobial peptide described above may be used as a therapeutic agent for plants infected with and/or at risk of being infected by Candidatus Liberibacte asiaticus (CLas).

Additional aspects of the inventive technology may include embodiments wherein the composition described above may be topically applied to plants infected with and/or at risk of being infected by CLas.

Additional aspects of the inventive technology may include embodiments wherein the composition described above may be used as a therapeutic agent for the treatment and/or prevention of Huanglongbing (HLB).

Additional aspects of the inventive technology may include embodiments wherein the antimicrobial peptide described above has two amphipathic helical peptides coupled by a linker domain forming a helix-turn-helix scaffold formation and wherein at least one hydrophobic amino acid residue from each of the amphipathic helical peptides are replaced with a cysteine residue forming a disulfide bridge between the amphipathic helical peptides and has increased bactericidal effects compared to a single endogenous amphipathic helical peptide.

Additional aspects of the inventive technology may include embodiments wherein the antimicrobial peptide described above has two amphipathic helical peptides coupled by a linker domain forming a helix-turn-helix scaffold formation and wherein at least one hydrophobic amino acid residue from each of the amphipathic helical peptides are replaced with a cysteine residue forming a disulfide bridge between the amphipathic helical peptides and has increased efficiency of attachment and/or insertion into a bacterial membrane compared to a single endogenous amphipathic helical peptide.

Additional aspects of the inventive technology may include embodiments wherein the antimicrobial peptide described above has two amphipathic helical peptides coupled by a linker domain forming a helix-turn-helix scaffold formation and wherein at least one hydrophobic amino acid residue from each of the amphipathic helical peptides are replaced with a cysteine residue forming a disulfide bridge between the amphipathic helical peptides has a lower susceptibility to bacterial resistance compared to a single endogenous amphipathic helical peptide.

Additional aspects of the inventive technology may include embodiments wherein the antimicrobial peptide described above may further comprise a second linker domain coupling the two P11 amphipathic helical peptides forming a cyclic scaffold formation identified as amino acid SEQ ID NO. 11.

Another aspect of the current inventive technology may include a novel antimicrobial peptide comprising a first amphipathic helical peptide and a second amphipathic helical peptide coupled by a first and a second linker domain forming a cyclic scaffold formation.

Additional aspects of the inventive technology may include the antimicrobial peptide described above wherein a first amphipathic helical peptide and a second amphipathic helical peptide are both endogenous amphipathic helical peptides from a citrus plant.

Additional aspects of the inventive technology may include the antimicrobial peptide described above wherein a first amphipathic helical peptide and a second amphipathic helical peptide are each selected from the group consisting of: P11, 11P1, 12P, 12P1, 12P-2, 10P, and 27P, or any combination thereof.

Additional aspects of the inventive technology may include the antimicrobial peptide described above wherein an amphipathic helical peptide and a second amphipathic helical peptide are each selected from the group consisting of: SEQ ID NO. 1-2, 13-15, 19, and 21, or any combination thereof.

Additional aspects of the inventive technology may include the antimicrobial peptide described above wherein the first and the second linker domains comprise a first and a second peptide linker having at least four amino acids respectively.

Additional aspects of the inventive technology may include the antimicrobial peptide described above wherein the first and the second linker domains comprise GPGR-turns having an amino acid sequence identified as SEQ ID NO. 23.

Additional aspects of the inventive technology may include the antimicrobial peptide described above wherein a first amphipathic helical peptide and a second amphipathic helical peptide are the same amphipathic helical peptide.

Additional aspects of the inventive technology may include the antimicrobial peptide described above wherein the antimicrobial peptide is identified as amino acid SEQ ID NO. 11.

Additional aspects of the inventive technology may include embodiments wherein the antimicrobial peptide described above is encoded by a polynucleotide comprising a nucleic acid sequence.

Additional aspects of the inventive technology may include the polynucleotide described above linked to a promoter to produce an expression vector.

Additional aspects of the inventive technology may include a genetically altered plant or plant cell comprising the polynucleotide described above operably linked to a promotor, wherein the plant or plant cell produce the antimicrobial peptide. In a preferred aspect, such a plant or plant cell may include a citrus plant or citrus plant cell.

Additional aspects of the inventive technology may include embodiments wherein the antimicrobial peptide described above may be a therapeutic agent for plants infected with and/or at risk of being infected by a bacterial pathogen.

Additional aspects of the inventive technology may include embodiments wherein the antimicrobial peptide described above may be used as a therapeutic agent for plants infected with and/or at risk of being infected by Candidatus Liberibacte asiaticus (CLas).

Additional aspects of the inventive technology may include embodiments wherein the composition described above may be topically applied to plants infected with and/or at risk of being infected by CLas.

Additional aspects of the inventive technology may include embodiments wherein the composition described above may be used as a therapeutic agent for the treatment and/or prevention of Huanglongbing (HLB).

Additional aspects of the inventive technology may include embodiments wherein the antimicrobial peptide described above has two P11 amphipathic helical peptides coupled by a first and a second linker domain forming a cyclic scaffold formation and has increased bactericidal effects compared to a single endogenous amphipathic helical peptide.

Additional aspects of the inventive technology may include embodiments wherein the antimicrobial peptide described above has two P11 amphipathic helical peptides coupled by a first and a second linker domain forming a cyclic scaffold formation having increased efficiency of attachment and/or insertion into a bacterial membrane compared to a single endogenous amphipathic helical peptide.

Additional aspects of the inventive technology may include embodiments wherein the antimicrobial peptide described above has two P11 amphipathic helical peptides coupled by a first and a second linker domain forming a cyclic scaffold formation and has a lower susceptibility to bacterial resistance compared to a single endogenous amphipathic helical peptide.

Additional aspects of the inventive technology may include the antimicrobial peptide described above wherein at least one hydrophobic amino acid residue from each of the amphipathic helical peptides are replaced with a cysteine residue forming a disulfide bridge between amphipathic helical peptides.

Another aspect of the current inventive technology may include a novel antimicrobial peptide comprising two P11 amphipathic helical peptides coupled by a first and a second linker domain forming a cyclic scaffold formation identified as amino acid SEQ ID NO. 11.

Additional aspects of the inventive technology may include the antimicrobial peptide described above wherein the P11 amphipathic helical peptides are both endogenous P11 amphipathic helical peptides from a citrus plant.

Additional aspects of the inventive technology may include the antimicrobial peptide described above wherein the linker domain comprises a peptide linker having at least four amino acids.

Additional aspects of the inventive technology may include the antimicrobial peptide described above wherein the linker domain comprises a GPGR-turn having an amino acid sequence identified as SEQ ID NO. 23.

Additional aspects of the inventive technology may include the antimicrobial peptide described above encoded by a polynucleotide comprising a nucleic acid sequence.

Additional aspects of the inventive technology may include the polynucleotide described above linked to a promoter to produce an expression vector.

Additional aspects of the inventive technology may include a genetically altered plant or plant cell comprising the polynucleotide described above operably linked to a promotor, wherein the plant or plant cell produce the antimicrobial peptide. In a preferred aspect, such a plant or plant cell may include a citrus plant or citrus plant cell.

Additional aspects of the inventive technology may include the use of the antimicrobial peptide described above as a therapeutic agent for plants infected with and/or at risk of being infected by a bacterial pathogen.

Additional aspects of the inventive technology may include use of the antimicrobial peptide described above as a therapeutic agent for plants infected with and/or at risk of being infected by Candidatus Liberibacte asiaticus (CLas).

Additional aspects of the inventive technology may include use of the composition described above as a topical application for plants infected with and/or at risk of being infected by CLas.

Additional aspects of the inventive technology may include use of the composition described above for use as a therapeutic agent for the treatment and/or prevention of

Huanglongbing (HLB).

Additional aspects of the inventive technology may include the antimicrobial peptide described above wherein the two P11 amphipathic helical peptides coupled by a first and a second linker domain forming a cyclic scaffold formation has increased bactericidal effects compared to a single endogenous amphipathic helical peptide.

Additional aspects of the inventive technology may include the antimicrobial peptide described above wherein the two P11 amphipathic helical peptides coupled by a first and a second linker domain forming a cyclic scaffold formation having increased efficiency of attachment and/or insertion into a bacterial membrane compared to a single endogenous amphipathic helical peptide.

Additional aspects of the inventive technology may include the antimicrobial peptide described above wherein the two P11 amphipathic helical peptides coupled by a first and a second linker domain forming a cyclic scaffold formation has a lower susceptibility to bacterial resistance compared to a single endogenous amphipathic helical peptide.

Additional aspects of the inventive technology may include the antimicrobial peptide described above wherein at least one hydrophobic amino acid residue from each of the P11 amphipathic helical peptides are replaced with a cysteine residue forming a disulfide bridge between the P11 amphipathic helical peptides.

Another aspect of the current inventive technology may include a novel antimicrobial peptide having a first amphipathic helical peptide and a second amphipathic helical peptide coupled by a linker domain forming a helix-turn-helix scaffold formation, the antimicrobial peptide comprising amino acid SEQ ID NO. 3.

Additional aspects of the inventive technology may include the novel antimicrobial peptide described above, for use as a therapeutic agent for the treatment and/or prevention of Huanglongbing (HLB).

Additional aspects of the inventive technology may include the novel antimicrobial peptide described above, for use as a topical therapeutic agent for citrus plants infected with and/or at risk of being infected by CLas.

Additional aspects of the inventive technology may include the antimicrobial peptide described above for use in a method of treating citrus plants infected with and/or at risk of being infected by CLas comprising the steps of: applying the composition described above to a citrus plant infected with and/or at risk of being infected by CLas.

Another aspect of the current inventive technology may include a novel antimicrobial peptide having a first amphipathic helical peptide and a second amphipathic helical peptide coupled by a linker domain forming a helix-turn-helix scaffold formation, the antimicrobial peptide comprising amino acid SEQ ID NO. 4.

Additional aspects of the inventive technology may include the novel antimicrobial peptide described above, for use as a therapeutic agent for the treatment and/or prevention of Huanglongbing (HLB).

Additional aspects of the inventive technology may include the novel antimicrobial peptide described above, for use as a topical therapeutic agent for citrus plants infected with and/or at risk of being infected by CLas.

Additional aspects of the inventive technology may include the antimicrobial peptide described above for use in a method of treating citrus plants infected with and/or at risk of being infected by CLas comprising the steps of: applying the composition described above to a citrus plant infected with and/or at risk of being infected by CLas.

Another aspect of the current inventive technology may include a novel antimicrobial peptide having a first amphipathic helical peptide and a second amphipathic helical peptide coupled by a linker domain forming a helix-turn-helix scaffold formation, the antimicrobial peptide comprising amino acid SEQ ID NO. 5.

Additional aspects of the inventive technology may include the novel antimicrobial peptide described above, for use as a therapeutic agent for the treatment and/or prevention of Huanglongbing (HLB).

Additional aspects of the inventive technology may include the novel antimicrobial peptide described above, for use as a topical therapeutic agent for citrus plants infected with and/or at risk of being infected by CLas.

Additional aspects of the inventive technology may include the antimicrobial peptide described above for use in a method of treating citrus plants infected with and/or at risk of being infected by CLas comprising the steps of: applying the composition described above to a citrus plant infected with and/or at risk of being infected by CLas.

Another aspect of the current inventive technology may include a novel antimicrobial peptide having a first amphipathic helical peptide and a second amphipathic helical peptide coupled by a linker domain forming a helix-turn-helix scaffold formation, the antimicrobial peptide comprising amino acid SEQ ID NO. 6.

Additional aspects of the inventive technology may include the novel antimicrobial peptide described above, for use as a therapeutic agent for the treatment and/or prevention of Huanglongbing (HLB).

Additional aspects of the inventive technology may include the novel antimicrobial peptide described above, for use as a topical therapeutic agent for citrus plants infected with and/or at risk of being infected by CLas.

Additional aspects of the inventive technology may include the antimicrobial peptide described above for use in a method of treating citrus plants infected with and/or at risk of being infected by CLas comprising the steps of: applying the composition described above to a citrus plant infected with and/or at risk of being infected by CLas.

Another aspect of the current inventive technology may include a novel antimicrobial peptide having a first amphipathic helical peptide and a second amphipathic helical peptide coupled by a linker domain forming a helix-turn-helix scaffold formation, the antimicrobial peptide comprising amino acid SEQ ID NO. 7.

Additional aspects of the inventive technology may include the novel antimicrobial peptide described above, for use as a therapeutic agent for the treatment and/or prevention of Huanglongbing (HLB).

Additional aspects of the inventive technology may include the novel antimicrobial peptide described above, for use as a topical therapeutic agent for citrus plants infected with and/or at risk of being infected by CLas.

Additional aspects of the inventive technology may include the antimicrobial peptide described above for use in a method of treating citrus plants infected with and/or at risk of being infected by CLas comprising the steps of: applying the composition described above to a citrus plant infected with and/or at risk of being infected by CLas.

Another aspect of the current inventive technology may include a novel antimicrobial peptide having a first amphipathic helical peptide and a second amphipathic helical peptide coupled by a linker domain forming a helix-turn-helix scaffold formation, the antimicrobial peptide comprising amino acid SEQ ID NO. 8.

Additional aspects of the inventive technology may include the novel antimicrobial peptide described above, for use as a therapeutic agent for the treatment and/or prevention of Huanglongbing (HLB). Additional aspects of the inventive technology may include the novel antimicrobial peptide described above, for use as a topical therapeutic agent for citrus plants infected with and/or at risk of being infected by CLas.

Additional aspects of the inventive technology may include the antimicrobial peptide described above for use in a method of treating citrus plants infected with and/or at risk of being infected by CLas comprising the steps of: applying the composition described above to a citrus plant infected with and/or at risk of being infected by CLas.

Another aspect of the current inventive technology may include a novel antimicrobial peptide having a first amphipathic helical peptide and a second amphipathic helical peptide coupled by a linker domain forming a helix-turn-helix scaffold formation stabilized by at least one disulfide bridge between a first amphipathic helical peptide and a second amphipathic helical peptide, the antimicrobial peptide comprising SEQ ID NO. 9.

Additional aspects of the inventive technology may include the novel antimicrobial peptide described above, for use as a therapeutic agent for the treatment and/or prevention of Huanglongbing (HLB).

Additional aspects of the inventive technology may include the novel antimicrobial peptide described above, for use as a topical therapeutic agent for citrus plants infected with and/or at risk of being infected by CLas.

Additional aspects of the inventive technology may include the antimicrobial peptide described above for use in a method of treating citrus plants infected with and/or at risk of being infected by CLas comprising the steps of: applying the composition described above to a citrus plant infected with and/or at risk of being infected by CLas.

Another aspect of the current inventive technology may include a novel antimicrobial peptide having a first amphipathic helical peptide and a second amphipathic helical peptide coupled by a linker domain forming a helix-turn-helix scaffold formation, the antimicrobial peptide comprising amino acid SEQ ID NO. 10

Additional aspects of the inventive technology may include the novel antimicrobial peptide described above, for use as a therapeutic agent for the treatment and/or prevention of Huanglongbing (HLB).

Additional aspects of the inventive technology may include the novel antimicrobial peptide described above, for use as a topical therapeutic agent for citrus plants infected with and/or at risk of being infected by CLas.

Additional aspects of the inventive technology may include the antimicrobial peptide described above for use in a method of treating citrus plants infected with and/or at risk of being infected by CLas comprising the steps of: applying the composition described above to a citrus plant infected with and/or at risk of being infected by CLas.

Another aspect of the current inventive technology may include a novel antimicrobial peptide having a first amphipathic helical peptide and a second amphipathic helical peptide coupled by a first and a second linker domain forming a cyclic scaffold formation, the antimicrobial peptide comprising amino acid SEQ ID NO. 11.

Additional aspects of the inventive technology may include the novel antimicrobial peptide described above, for use as a therapeutic agent for the treatment and/or prevention of Huanglongbing (HLB).

Additional aspects of the inventive technology may include the novel antimicrobial peptide described above, for use as a topical therapeutic agent for citrus plants infected with and/or at risk of being infected by CLas.

Additional aspects of the inventive technology may include the antimicrobial peptide described above for use in a method of treating citrus plants infected with and/or at risk of being infected by CLas comprising the steps of: applying the composition described above to a citrus plant infected with and/or at risk of being infected by CLas.

Another aspect of the current inventive technology may include a novel antimicrobial peptide having a first amphipathic helical peptide and a second amphipathic helical peptide coupled by a linker domain forming a helix-turn-helix scaffold formation, the antimicrobial peptide comprising amino acid SEQ ID NO. 12.

Additional aspects of the inventive technology may include the novel antimicrobial peptide described above, for use as a therapeutic agent for the treatment and/or prevention of Huanglongbing (HLB).

Additional aspects of the inventive technology may include the novel antimicrobial peptide described above, for use as a topical therapeutic agent for citrus plants infected with and/or at risk of being infected by CLas.

Additional aspects of the inventive technology may include the antimicrobial peptide described above for use in a method of treating citrus plants infected with and/or at risk of being infected by CLas comprising the steps of: applying the composition described above to a citrus plant infected with and/or at risk of being infected by CLas.

Another aspect of the current inventive technology may include a novel antimicrobial peptide having a first amphipathic helical peptide and a second amphipathic helical peptide coupled by a linker domain forming a helix-turn-helix scaffold formation, the antimicrobial peptide comprising amino acid SEQ ID NO. 16.

Additional aspects of the inventive technology may include the novel antimicrobial peptide described above, for use as a therapeutic agent for the treatment and/or prevention of Huanglongbing (HLB).

Additional aspects of the inventive technology may include the novel antimicrobial peptide described above, for use as a topical therapeutic agent for citrus plants infected with and/or at risk of being infected by CLas.

Additional aspects of the inventive technology may include the antimicrobial peptide described above for use in a method of treating citrus plants infected with and/or at risk of being infected by CLas comprising the steps of: applying the composition described above to a citrus plant infected with and/or at risk of being infected by CLas.

Another aspect of the current inventive technology may include a novel antimicrobial peptide having a first amphipathic helical peptide and a second amphipathic helical peptide coupled by a linker domain forming a helix-turn-helix scaffold formation, the antimicrobial peptide comprising amino acid SEQ ID NO. 17.

Additional aspects of the inventive technology may include the novel antimicrobial peptide described above, for use as a therapeutic agent for the treatment and/or prevention of Huanglongbing (HLB).

Additional aspects of the inventive technology may include the novel antimicrobial peptide described above, for use as a topical therapeutic agent for citrus plants infected with and/or at risk of being infected by CLas.

Additional aspects of the inventive technology may include the antimicrobial peptide described above for use in a method of treating citrus plants infected with and/or at risk of being infected by CLas comprising the steps of: applying the composition described above to a citrus plant infected with and/or at risk of being infected by CLas.

Another aspect of the current inventive technology may include a novel antimicrobial peptide having a first amphipathic helical peptide and a second amphipathic helical peptide coupled by a linker domain forming a helix-turn-helix scaffold formation, the antimicrobial peptide comprising amino acid SEQ ID NO. 18.

Additional aspects of the inventive technology may include the novel antimicrobial peptide described above, for use as a therapeutic agent for the treatment and/or prevention of Huanglongbing (HLB).

Additional aspects of the inventive technology may include the novel antimicrobial peptide described above, for use as a topical therapeutic agent for citrus plants infected with and/or at risk of being infected by CLas.

Additional aspects of the inventive technology may include the antimicrobial peptide described above for use in a method of treating citrus plants infected with and/or at risk of being infected by CLas comprising the steps of: applying the composition described above to a citrus plant infected with and/or at risk of being infected by CLas.

Another aspect of the current inventive technology may include a novel antimicrobial peptide having a first amphipathic helical peptide and a second amphipathic helical peptide coupled by a linker domain forming a helix-turn-helix scaffold formation, the antimicrobial peptide comprising amino acid SEQ ID NO. 20.

Additional aspects of the inventive technology may include the novel antimicrobial peptide described above, for use as a therapeutic agent for the treatment and/or prevention of Huanglongbing (HLB).

Additional aspects of the inventive technology may include the novel antimicrobial peptide described above, for use as a topical therapeutic agent for citrus plants infected with and/or at risk of being infected by CLas.

Additional aspects of the inventive technology may include the antimicrobial peptide described above for use in a method of treating citrus plants infected with and/or at risk of being infected by CLas comprising the steps of: applying the composition described above to a citrus plant infected with and/or at risk of being infected by CLas.

Additional aspects of the inventive technology may include the novel antimicrobial peptide described above, for use as a therapeutic agent for the treatment and/or prevention of Huanglongbing (HLB).

Additional aspects of the inventive technology may include the novel antimicrobial peptide described above, for use as a topical therapeutic agent for citrus plants infected with and/or at risk of being infected by CLas.

Additional aspects of the inventive technology may include the antimicrobial peptide described above for use in a method of treating citrus plants infected with and/or at risk of being infected by CLas comprising the steps of: applying the composition described above to a citrus plant infected with and/or at risk of being infected by CLas.

Another aspect of the current inventive technology may include a novel antimicrobial peptide having a first amphipathic helical peptide and a second amphipathic helical peptide coupled by a linker domain forming a helix-turn-helix scaffold formation, the antimicrobial peptide comprising amino acid SEQ ID NO. 22.

Additional aspects of the inventive technology may include the novel antimicrobial peptide described above, for use as a therapeutic agent for the treatment and/or prevention of Huanglongbing (HLB).

Additional aspects of the inventive technology may include the novel antimicrobial peptide described above, for use as a topical therapeutic agent for citrus plants infected with and/or at risk of being infected by CLas.

Additional aspects of the inventive technology may include the antimicrobial peptide described above for use in a method of treating citrus plants infected with and/or at risk of being infected by CLas comprising the steps of: applying the composition described above to a citrus plant infected with and/or at risk of being infected by CLas.

Another aspect of the current inventive technology may include a novel method of predicting relative bactericidal activities of an antimicrobial peptide comprising the steps of: identifying an amphipathic helical peptide; generating a modified peptide consisting essentially of two of the amphipathic helical peptides coupled by a linker domain forming a helix-turn-helix scaffold formation; establishing lipid:water bilayer parameters to generate a simulated bacterial membrane; performing a molecular dynamics (MD) simulation to determine the relative efficiencies of the amphipathic helical peptide and the modified peptide to attach to the simulated bacterial membrane, or insert into the simulated bacterial membrane, or maintain their configuration after attachment or insertion; and comparing the relative bactericidal activity of the amphipathic helical peptide and the modified peptide.

Additional aspects of the inventive technology may include the method described above wherein the step of identifying a first amphipathic helical peptide comprises the step of identifying an amphipathic helical peptide that is endogenous to a plant.

Additional aspects of the inventive technology may include the method described above wherein the step of identifying an amphipathic helical peptide that is endogenous to a plant comprises the step of identifying an amphipathic helical peptide that is endogenous to a citrus plant.

Additional aspects of the inventive technology may include the method described above wherein the amphipathic helical peptide is a dimer.

Additional aspects of the inventive technology may include the method described above wherein the linker domain comprises a peptide linker having at least four amino acids.

Additional aspects of the inventive technology may include the method described above wherein the peptide linker having at least four amino acids comprises a GPGR-turn.

Additional aspects of the inventive technology may include the method described above and further comprising the step of applying a GROMOS force-field to monitor the attachment of the amphipathic helical peptide and the modified peptide from water to the lipid.

Additional aspects of the inventive technology may include the method described above wherein the step of establishing lipid:water bilayer parameters to generate a simulated bacterial membrane further comprises the step of establishing one of more parameters selected from the group consisting of: establishing the number of water molecules in the lipid core; establishing the number of polar lipid head groups flipped into the lipid core; establishing the fraction of residues in the hydrophobic core; and establishing the helical content.

Additional aspects of the inventive technology will be evident from the detailed description and figures presented below.

### BRIEF DESCRIPTION OF DRAWINGS

The novel aspects, features, and advantages of the present disclosure will be better understood from the following detailed descriptions taken in conjunction with the accompanying figures, all of which are given by way of illustration only, and are not limiting the presently disclosed embodiments, in which:

FIG. 1—Design and structure of novel antimicrobial peptides based on host analogs in one embodiment thereof.

FIG. 2—(A) Dimensions of water-lipid bilayer used in the Molecular Dynamics (MD); and (B) chemical structure and dimensions of the POPE:POPG.

FIG. 3—Comparison of (A) attachment and (B) insertion of P11 and P26 in one embodiment thereof.

FIG. 4—Percentage clearance of CLas by antimicrobial peptides from (left) infected citrus leaves and (right) infected psyllids. CK=negative control; Triton (0.1%)=positive control; TMOF=a psyllid gut-binding peptide (not specific for CLas clearance).

FIG. 5—(A) and (B). Percentage of hemolysis is shown for different exemplary antimicrobial peptides at different concentrations

FIG. 6—(A) Helix-turn-helix, P26, engineered by joining two endogenous P11 by a turn. (B) A detached leaf assay showing that P26 is more active than streptomycin. In P11-R, all basic Ks are replaced by R.

FIG. 7—Sequence and structural helix-turn-helix motifs for exemplary engineered antimicrobial peptides: P26, cysP26, and P30.

FIG. 8—Data set demonstrating the MIC and toxicity effect in human cell lines, in particular erythrocyte, HL60 cells, as well as measurements demonstrating low phytotoxcicity for exemplary engineered antimicrobial peptides: P26, cysP26, and P30.

FIG. 9—Effect of different peptides on the viability of N. benthamiana mesophyll protoplasts demonstrating low phytotoxcicity of P26 and cysP26.

### DETAILED DESCRIPTION OF INVENTION

The present invention includes a variety of aspects, which may be combined in different ways. The following descriptions are provided to list elements and describe some of the embodiments of the present invention. These elements are listed with initial embodiments, however it should be understood that they may be combined in any manner and in any number to create additional embodiments. The variously described examples and preferred embodiments should not be construed to limit the present invention to only the explicitly described systems, techniques, and applications. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

Disclosed herein are novel systems, methods, and compositions for the treatment of bacterial infections in plants. These inventions may further include novel systems, methods, and compositions for the treatment of gram-negative bacterial infections in plants. In one specific embodiment, the invention may include novel systems, methods, and compositions for the treatment of HLB disease, preferably in citrus plants. In this embodiment, the invention may include novel antimicrobial peptides that may be used to treat susceptible or already infected citrus plants, which may cure, or lower the bacterial load and increase the productive years of the citrus plants. Additional embodiments may include the generation of transgenic HLB-resistant citrus plants that express one or more of the antimicrobial peptides described herein for long-term disease protection.

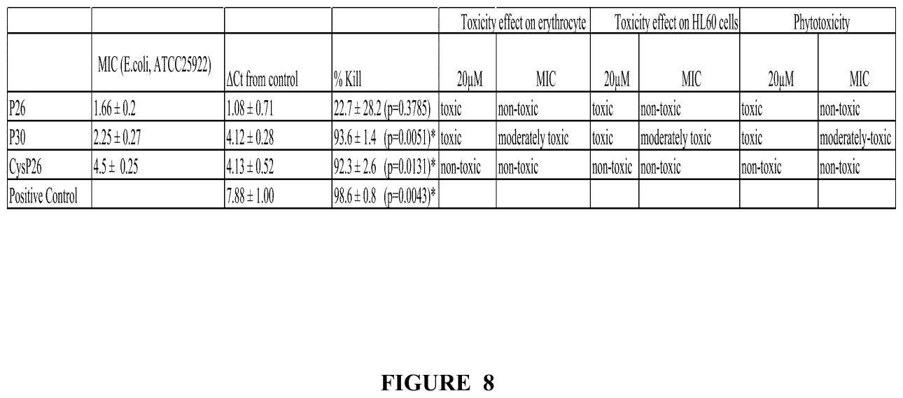

The invention may include engineered antimicrobial peptides to treat HLB disease, preferably in citrus plants. In this embodiment, the invention may include novel antimicrobial peptide derived from amphipathic helical peptides that may further be used to treat HLB disease in citrus plants. In one embodiment, the invention may include an engineered antimicrobial peptide formed by coupling two amphipathic helical peptides. Specifically, a generalized antimicrobial peptide of the invitation may include a first amphipathic helical peptide coupled with a second amphipathic helical peptide by a linker domain forming a helix-turn-helix scaffold formation. Such amphipathic helical peptides may be endogenous to a target host, preferably a citrus plant. In another preferred embodiment, such engineered antimicrobial peptides may be non-toxic to a cell host. In this embodiment, engineered antimicrobial peptides may be non-toxic in human. For example, as shown in FIG. 8, exemplary engineered antimicrobial peptides P26 and cysP26 did not demonstrate toxicity to human erythrocytes or HL60 cells.

As noted above, a variety of endogenous single amphipathic helical peptides may be generated by plants and other organisms to defend against bacterial infections. As such, in one embodiment, the invention may include an antimicrobial peptide having a first amphipathic helical peptide and a second amphipathic helical peptide coupled by a linker domain forming a helix-turn-helix scaffold formation where each amphipathic helical peptide may be selected from the group of amphipathic helical peptides that may be endogenous to a host plant, such as a citrus plant. In one embodiment, a first amphipathic helical peptide and a second amphipathic helical peptide may each be selected from the group of amphipathic helical peptides consisting of: P11, 11P1, 12P, 12P1, 12P-2, 10P, and 27P, or any combination thereof.

For example, in one preferred embodiment the invention may include an antimicrobial peptide having a first P11 amphipathic helical peptide and a second P11 amphipathic helical peptide coupled by a linker domain forming a helix-turn-helix scaffold formation. Additional embodiments may include additional identical, as well as non-identical combinations thereof and as such, should not be considered limiting on the broad scope of combinations of amphipathic helical peptides contemplated within the scope of the invention.

In one additional embodiment, the invention may include an antimicrobial peptide having a first amphipathic helical peptide and a second amphipathic helical peptide coupled by a linker domain forming a helix-turn-helix scaffold formation where the amphipathic helical peptides are the same. In this embodiment, the invention may include an antimicrobial peptide selected from the group consisting of: P26, 26P1, 26P2, 26P3, 26P4, 26P5, cysP30, 41P, 28P, 28P1, 28P1-2, 24P, and 58-P as generally described herein. It should be noted that the terms “same” or “identical” as used throughout may include amphipathic helical peptides having identical or similar sequences, structures, or identical designations, as well as homologous sequences or structures as defined herein.

In another embodiment, the invention may include an antimicrobial peptide having a first peptide and a second peptide coupled by a linker domain forming a helix-turn-helix scaffold formation where each amphipathic helical peptide may each be selected from the group of amino acid sequences consisting of: SEQ ID NOs. 1-2, 13-15, 19, and 21, or any combination thereof. For example, in one preferred embodiment, the invention may include an antimicrobial peptide having a first amphipathic helical peptide, identified as SEQ ID NO. 3, and a second amphipathic helical peptide, identified as SEQ ID NO. 3, coupled by a linker domain forming a helix-turn-helix scaffold formation. Additional embodiments may include additional identical, as well as non-identical combinations of amino acid sequences thereof and as such, should not be considered limiting on the broad scope of combinations of amino acid sequences contemplated within the scope of the invention.

In one additional embodiment, the invention may include an antimicrobial peptide having a first amphipathic peptide and a second amphipathic peptide coupled by a linker domain forming a helix-turn-helix scaffold formation where the first and second peptides sequences are the same. In this embodiment, the invention may include an antimicrobial peptide selected from the group consisting of: SEQ ID NOs. 3-12, 16-18, 20, and 22-23, or a variant thereof as generally described herein. Such variants may include sequences having approximately between 75% to 99% sequence homology.

As noted above, a linker domain may couple together a first and second amphipathic helical peptide. In one embodiment, this linker domain may include an amino acid sequence configured to generate the “turn” in a helix-turn-helix scaffold formation as generally described herein. This linker domain may include a peptide linker having at least four amino acids. In a preferred embodiment, this linker domain may include a GPGR-turn having an amino acid sequence identified as SEQ ID NO. 23.

As noted above, the invention may include one or more of the antimicrobial peptides identified herein to treat bacterial infections in plants. For example, the invention may include one or more of the antimicrobial peptides described herein as a therapeutic agent for plants infected with and/or at risk of being infected by a bacterial pathogen, preferably a gram-negative bacterial pathogen. In this embodiment, one or more of the antimicrobial peptides identified herein may be used a therapeutic agent for plants infected with and/or at risk of being infected by Candidatus Liberibacte asiaticus (CLas), a causative agent of HLB disease.

In one specific example, an antimicrobial peptide, P26 identified as amino acid SEQ ID NO. 3, may include a first P11 amphipathic helical peptide and a second P11 amphipathic helical peptide coupled by a linker domain forming a helix-turn-helix scaffold formation. This engineered P26 antimicrobial peptide may be a therapeutic agent for plants, and more specifically citrus plants infected with and/or at risk of being infected by Candidatus Liberibacte asiaticus (CLas). In this embodiment, such engineered P26 antimicrobial peptide may exhibit a therapeutic effect against CLas, or other gram-negative bacteria through an enhanced bactericidal effect as compared to a single endogenous amphipathic helical peptide P11 sub-component. More specifically, such engineered P26 antimicrobial peptide may exhibit increased efficiency of attachment and/or insertion into a bacterial membrane compared to an endogenous single amphipathic helical peptide P11 sub-component. In this configuration, the engineered P26 antimicrobial peptide may more efficiently attach to, and insert itself into the bacterial membrane of a gram-negative bacterial pathogen, such as CLas, causing lysis of the bacteria. Moreover, as described elsewhere, due to the structure of the novel helix-turn-helix scaffold structure, and its more efficient bactericidal profile, such an exemplary engineered P26 antimicrobial peptide may exhibit lower susceptibility to bacterial resistance and protease degradation compared to a single P11 endogenous amphipathic helical peptide sub-component.

In one preferred embodiment, this engineered P26 antimicrobial peptide may form a composition that may be administered to plants, and more specifically citrus plants infected with and/or at risk of being infected by CLas. In this manner, an exemplary P26 antimicrobial peptide may be administered to a plant in need thereof as a therapeutic agent for the treatment and/or prevention of HLB disease. In this preferred embodiment, the exemplary P26 antimicrobial peptide may be topically administered as a composition to a plant in need thereof as a therapeutic agent for the treatment and/or prevention of HLB disease.

The invention may include a novel antimicrobial peptide having two amphipathic helical peptides coupled by a linker domain forming a helix-turn-helix scaffold formation and further modified such that at least one hydrophobic amino acid residue from each of the amphipathic helical peptides are replaced with a cysteine residue forming a disulfide bridge between the amphipathic helical peptides. In this embodiment, the disulfide bridge may stabilize or reinforce the helix-turn-helix scaffold formation such that it may exhibit enhanced bactericidal activity, as well as increased stability and resistance to protease degradation. Such amphipathic helical peptides may be endogenous to, or derived from a target host, preferably a citrus plant.

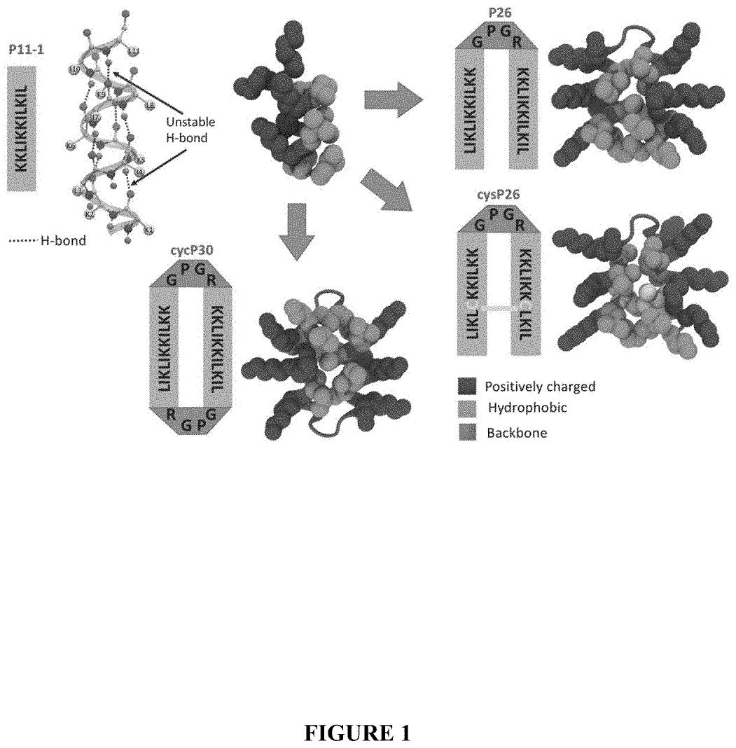

Such a disulfide bridge stabilized antimicrobial peptide may be used to treat bacterial infections and their associated conditions. In one embodiment, a disulfide bridge stabilized antimicrobial peptide as generally described herein may be used to treat HLB disease, preferably in citrus plants. As noted above, a variety of endogenous single amphipathic helical peptides may be generated by plants and other organisms to defend against bacterial infections. As such, in one embodiment, the invention may include an antimicrobial peptide having a first amphipathic helical peptide and a second amphipathic helical peptide coupled by a linker domain forming a helix-turn-helix scaffold formation where each amphipathic helical peptide may be selected from the group of endogenous amphipathic helical peptide consisting of: P11, 11P1, 12P, 12P1, 12P-2, 10P, and 27P, or any combination thereof, and wherein at least one hydrophobic amino acid residue from each of the amphipathic helical peptides are replaced with a cysteine residue forming a disulfide bridge between the amphipathic helical peptides. For example, in a specific preferred embodiment, the invention may include an antimicrobial peptide having a first P11 amphipathic helical peptide and a second P11 amphipathic helical peptide coupled by a linker domain forming a helix-turn-helix scaffold formation wherein at least one hydrophobic amino acid residue from each of the amphipathic helical peptides are replaced with a cysteine residue forming a disulfide bridge between the amphipathic helical peptide as generally shown in FIG. 1. Additional embodiments may include additional identical, as well as non-identical combinations thereof and as such, should not be considered limiting on the broad scope of combinations of amphipathic helical peptides that may be stabilized through a disulfide bridge contemplated within the scope of the invention.

In one additional embodiment, the invention may include an antimicrobial peptide having a first amphipathic helical peptide and a second amphipathic helical peptide coupled by a linker domain forming a helix-turn-helix scaffold formation wherein at least one hydrophobic amino acid residue from each of the amphipathic helical peptides are replaced with a cysteine residue forming a disulfide bridge between the amphipathic helical peptide where are the amphipathic helical peptides are the same. In this embodiment, the invention may include an antimicrobial peptide selected from the group consisting of: P26, 26P1, 26P2, 26P3, 26P4, 26P5, cysP30, 41P, 28P, 28P1, 28P1-2, 24P, and 58-P as generally described herein. In another embodiment, the invention may include an antimicrobial peptide having a first peptide and a second peptide coupled by a linker domain forming a helix-turn-helix scaffold formation where each amphipathic helical peptide may be selected from the group of amino acid sequences consisting of: SEQ ID NOs. 1-2, 13-15, 19, and 21, or any combination thereof wherein at least one hydrophobic amino acid residue from each of the amphipathic helical peptides are replaced with a cysteine residue forming a disulfide bridge between the amphipathic helical peptide.

In one specific preferred embodiment, an antimicrobial peptide comprising two P11 amphipathic helical peptides derived from a citrus plant may be coupled by a linker domain forming a helix-turn-helix scaffold formation identified as amino acid SEQ ID NO. 3 and may further be modified where at least one hydrophobic amino acid residue from each of the P11 amphipathic helical peptides are replaced with a cysteine residue forming a disulfide bridge between the P11 amphipathic helical peptides which may be identified as amino acid SEQ ID NO. 9. As generally described above, such an antimicrobial peptide having a disulfide bridge stabilized helix-turn-helix scaffold, identified as cysP26, may further include a linker domain that may couple the first and second P11 amphipathic helical peptides. In one embodiment, this linker domain may include an amino acid sequence configured to generate the “turn” in a helix-turn-helix scaffold formation as generally described herein. In this embodiment, this linker domain may include a peptide linker having at least four amino acids. In a preferred embodiment, this linker domain may include a GPGR-turn having an amino acid sequence identified as SEQ ID NOs. 23.

Moreover, in one specific example, the antimicrobial peptide cysP26, identified as amino acid SEQ ID NO.9, may be a therapeutic agent for plants, and more specifically citrus plants infected with and/or at risk of being infected by CLas. In this embodiment, such engineered cysP26 antimicrobial peptide may exhibit a therapeutic effect against CLas, or other gram-negative bacteria through an enhanced bactericidal effect as compared to a single endogenous amphipathic helical peptide P11 sub-component. More specifically, such engineered cysP26 antimicrobial peptide may exhibit increased efficiency of attachment and/or insertion into a bacterial membrane compared to an endogenous single amphipathic helical peptide P11 sub-component. In this configuration, the engineered cysP26 antimicrobial peptide may more efficiently attach to, and insert itself into the bacterial membrane of CLas causing lysis of the bacteria. Moreover, as described elsewhere, due to the structure of the invention's engineered helix-turn-helix scaffold structure that is further stabilized by a disulfide bridge between each amphipathic helical peptide, such an exemplary engineered cysP26 antimicrobial peptide may exhibit lower susceptibility to bacterial resistance compared to a single P11 endogenous amphipathic helical peptide as well as enhanced resistance to cellular protease degradation.

In one preferred embodiment, this engineered cysP26 antimicrobial peptide may form a composition that may be administered to plants, and more specifically citrus plants infected with and/or at risk of being infected by CLas. In this manner, an exemplary cysP26 antimicrobial peptide may be administered to a plant in need thereof as a therapeutic agent for the treatment and/or prevention of Huanglongbing (HLB). In this preferred embodiment, the exemplary cysP26 antimicrobial peptide composition may be topically administered to a plant in need thereof as a therapeutic agent for the treatment and/or prevention of Huanglongbing (HLB).

The invention may also include a novel antimicrobial peptide having a first amphipathic helical peptide and a second amphipathic helical peptide coupled by a first and a second linker domain forming a cyclic scaffold formation. In this embodiment, such cyclic scaffold formation may exhibit enhanced bactericidal activity, as well as increased stability and resistance to cellular proteases. Such modified amphipathic helical peptides may be endogenous to or derived from a target host, preferably a citrus plant. In additional embodiments, at least one hydrophobic amino acid residue from each of the amphipathic helical peptides may be replaced with a cysteine residue forming a disulfide bridge between the amphipathic helical peptides in the cyclic scaffold formation.

Such a cyclic scaffold formation antimicrobial peptide may be used to treat bacterial infections and their associated conditions in plants. In one embodiment, a cyclic scaffold formation antimicrobial peptide as generally described herein may be used to treat HLB disease, preferably in citrus plants. As noted above, a variety of endogenous single amphipathic helical peptides may be generated by plants and other organisms to defend against bacterial infections. As such, in one embodiment, the invention may include an antimicrobial peptide having a first amphipathic helical peptide and a second amphipathic helical peptide coupled by at least two linker domains forming a cyclic scaffold formation as generally shown in FIG. 1, where each amphipathic helical peptide may be selected from the group of endogenous amphipathic helical peptides consisting of: P11, 11P1, 12P, 12P1, 12P-2, 10P, and 27P, or any combination thereof.

For example, in one preferred embodiment, the invention may include an antimicrobial peptide having a first P11 amphipathic helical peptide and a second P11 amphipathic helical peptide coupled by two opposing linker domains forming a cyclic scaffold formation as generally shown in FIG. 1. As noted above, additional embodiments may include a first amphipathic helical peptide and a second amphipathic helical peptide coupled by at least two linker domains forming a cyclic scaffold formation where both amphipathic helical peptides are the same, while in alternative embodiments a first amphipathic helical peptide and a second amphipathic helical peptide may include non-identical combinations of amphipathic helical peptides. In another embodiment, the invention may include an antimicrobial peptide having a first peptide and a second peptide coupled by at least two linker domains forming a cyclic scaffold formation where each amphipathic helical peptide may be selected from the group of amino acid sequences consisting of: SEQ ID NOs. 1-2, 13-15, 19, and 21, or any combination thereof coupled with a second linker domain identified as SEQ ID NO. 23.

In one additional embodiment, the invention may include an antimicrobial peptide having a first P11 amphipathic helical peptide and a P11 second amphipathic helical peptide coupled by at least two linker domains forming a cyclic scaffold formation identified as cycP30 as generally shown in FIG. 1. In another specific embodiment, an antimicrobial peptide comprising two P11 amphipathic helical peptides derived from a citrus plant may be coupled by at least two linker domains forming a cyclic scaffold formation identified as amino acid SEQ ID NO. 11. As generally described above, such a cyclic scaffold formation antimicrobial peptide may further include a disulfide bridge stabilized cyclic scaffold formation as generally described herein. In one embodiment, these linker domains may include an amino acid sequence configured to generate the “turns” in a cyclic scaffold formation as generally described herein. In this embodiment, these linker domains may include peptide linkers having at least four amino acids. In a preferred embodiment, these linker domains may include a GPGR-turn having an amino acid sequence identified as SEQ ID NOs. 23.

Moreover, in one specific example, an antimicrobial peptide identified as cycP30, identified as amino acid SEQ ID NO.9, may be used as a therapeutic agent for plants, and more specifically citrus plants infected with and/or at risk of being infected by CLas. In this embodiment, such engineered cycP30 antimicrobial peptide may exhibit a therapeutic effect against CLas, or other gram-negative bacteria through an enhanced bactericidal effect as compared to a single endogenous amphipathic helical peptide P11 sub-component. More specifically, such engineered cycP30 antimicrobial peptide may exhibit increased efficiency of attachment and/or insertion into a bacterial membrane compared to an endogenous single amphipathic helical peptide P11 sub-component. In this configuration, the engineered cycP30 antimicrobial peptide may more efficiently attach to, and insert itself into the bacterial membrane of CLas causing lysis of the bacteria. Moreover, as described elsewhere, due to the structure of the invention's engineered cyclic scaffold formation structure, that may further be stabilized by a disulfide bridge between each amphipathic helical peptide, and its more efficient bactericidal profile, such an exemplary engineered cycP30 antimicrobial peptide may exhibit lower susceptibility to bacterial resistance compared to a single P11 endogenous amphipathic helical peptide as well as enhanced resistance to protease degradation.

In one preferred embodiment, this engineered cycP30 antimicrobial peptide may be administered to plants as a composition, and more specifically citrus plants infected with and/or at risk of being infected by CLas, or other gram-negative bacteria preferably. In this manner, an exemplary cycP30 antimicrobial peptide may be administered to a plant in need thereof as a therapeutic agent for the treatment and/or prevention of HLB disease. In this preferred embodiment, the exemplary cycP30 antimicrobial peptide may be topically administered as a composition to a plant in need thereof as a therapeutic agent for the treatment and/or prevention of HLB disease.

The term “applying,” “application,” “administering,” “administration,” and all their cognates, as used herein, refers to any method for contacting the plant with the antimicrobial peptide compositions discussed herein. Administration generally is achieved by application of the compositions in a vehicle compatible with the plant to be treated (i.e., a botanically compatible vehicle or carrier), such as an aqueous vehicle, to the plant or to the soil surrounding the plant or by injection into the plant. Any application can be used, however one application methods include trunk injection and foliar spraying as described herein. Other methods include application to the soil surrounding the plant, by injection, soaking or spraying, so that the applied compounds can come into contact with the plant roots and can be taken up by the roots. Additional topical applications may also be contemplated. The compositions disclosed herein can be formulated for seed or plant treatments in any of the following modes: dry powder, water slurriable powder, liquid solution, flowable concentrate or emulsion, emulsion, microcapsules, gel, or water dispersible granules.

The antimicrobial peptide compositions described herein can also be chosen from a number of formulation types, including isolated antimicrobial peptides, which may further be compled with dustable powders (DP), soluble powders (SP), water soluble granules (SG), water dispersible granules (WG), wettable powders (WP), granules (GR) (slow or fast release), soluble concentrates (SL), oil miscible liquids (OL), ultra-low volume liquids (UL), emulsifiable concentrates (EC), dispersible concentrates (DC), emulsions (both oil in water (EW) and water in oil (EO)), micro-emulsions (ME), suspension concentrates (SC), oil-based suspension concentrate (OD), aerosols, fogging/smoke formulations, capsule suspensions (CS) and seed/plant treatment formulations.

In another embodiment, delivery of the antimicrobial peptide composition to plants can be via different routes. The compositions can be suitably administered as an aerosol, for example by spraying onto leaves or other plant material. The particles can also be administered by injection, for example directly into a plant, such as into the stem. In certain embodiments the compositions are administered to the roots. This can be achieved by spraying or watering plant roots with compositions. In other embodiments, the particles are introduced into the xylem or phloem, for example by injection or being included in a water supply feeding the xylem or phloem. Application to the stems or leaves of the plant can be performed by spraying or other direct application to the desired area of the plant; however any method known in the art can be used. A solution or vehicle containing the antimicrobial peptides at a dosage of active ingredient can be applied with a sprayer to the stems or leaves until runoff to ensure complete coverage, and repeat three or four times in a growing season. The concentrations, volumes and repeat treatments may change depending on the plant.

Additional embodiments of the invention include a polynucleotide comprising a nucleic acid sequence that may encode one or more of the antimicrobial peptides described herein. In one specific example, the invention may include a polynucleotide comprising a nucleic acid sequence identified as SEQ ID NOs. 3-12, 16-18, 20, and 22-23, or a variant thereof. Such sequences may further be operably linked to a promotor to generate an expression vectors and further introduced to a plant, preferably a citrus plant. In this embodiment, such transformed plant or plant cell may produce the antimicrobial peptide. Such a transformed plant, which in a preferred embodiment may include a citrus plant, may exhibit enhanced resistance to Clas, a causative agent of HLB disease. In additional embodiment, a transformed citrus plant may exhibit decreased bacterial loads of Clas, and/or decreased symptoms or progression of HLB. Methods, systems and techniques of stable and transient plant transformation, such as Agrobacterium tumefaciens-mediated transformation, are know in the art and included within the scope of the inventive technology.

Another embodiment of the current inventive technology may include a novel method of predicting relative bactericidal activities of an antimicrobial peptide. In one embodiment, the invention may include novel water:lipid molecular dynamics (MD) simulation system that provides a method of predicting relative bactericidal activities of helix-turn-helix scaffold based upon host single helices among others. The MD simulation in water:lipid system as described herein may further predict relative efficiencies of peptides in terms of their ability to attach and insert into the bacterial membrane. Notably, the higher the efficiency of attachment and insertion, the higher is the bactericidal activity and the lower is the susceptibility to resistance. Regardless of how bacteria evolve resistance against single helices, helix-turn-helix engineering by MD simulation provides bactericides that are highly active and yet not susceptible to resistance

In this embodiment, the method may include the steps of: identifying an amphipathic helical peptide, such as for example P11 that is endogenous to a citrus plant. Next, the method may include the step of generating a modified peptide consisting essentially of two of the amphipathic helical peptides coupled by a linker domain forming a helix-turn-helix scaffold formation, such as P26 as described above. Next, the method may include establishing lipid:water bilayer parameters to generate a simulated bacterial membrane and them performing a molecular dynamics (MD) simulation to determine the relative efficiencies of the amphipathic helical peptide and the modified peptide to attach to a simulated bacterial membrane, or insert into a simulated bacterial membrane, or maintain their configuration after attachment or insertion; and comparing the relative bactericidal activity of the amphipathic helical peptide and the modified peptide. In additional embodiment, the amphipathic helical peptide, such as a P11 that is endogenous in a citrus plant may be evaluated as a dimer configuration.

Additional embodiments of the method may further comprise the step of applying a GROMOS force-field to monitor the attachment of the amphipathic helical peptide and the modified peptide from water to a lipid. Moreover, lipid:water bilayer parameters may be established to generate a simulated bacterial membrane which may include, but not be limited to: establishing the number of water molecules in the lipid core; establishing the number of polar lipid head groups flipped into the lipid core; establishing the fraction of residues in the hydrophobic core; and establishing the helical content.

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

The term, “antimicrobial peptide,” as used herein refers to any peptide that has microbiocidal and/or microbiostatic activity.

As used herein, a compound is referred to as “isolated” when it has been separated from at least one component with which it is naturally associated. For example, a metabolite can be considered isolated if it is separated from contaminants including polypeptides, polynucleotides and other metabolites. Isolated molecules can be either prepared synthetically or purified from their natural environment. Standard quantification methodologies known in the art can be employed to obtain and isolate the molecules of the invention.

The term “expression,” as used herein, or “expression” of a coding sequence (for example, a gene or a transgene) refers to the process by which the coded information of a nucleic acid transcriptional unit (including, e.g., genomic DNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

The term “nucleic acid” or “nucleic acid molecules” include single- and double-stranded forms of DNA; single-stranded forms of RNA; and double-stranded forms of RNA (dsRNA). The term “nucleotide sequence” or “nucleic acid sequence” refers to both the sense and antisense strands of a nucleic acid as either individual single strands or in the duplex.

The term “gene” or “sequence” refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (down-stream) the coding region (open reading frame, ORF) as well as, where applicable, intervening sequences (i.e., introns) between individual coding regions (i.e., exons).

A nucleic acid molecule may include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hairpinned, circular, and padlocked conformations.

The term “sequence identity” or “identity,” as used herein in the context of two nucleic acid or polypeptide sequences, refers to the residues in the two sequences that are the same when aligned for maximum correspondence over a specified comparison window. As used herein, the term “percentage of sequence identity” may refer to the value determined by comparing two optimally aligned sequences (e.g., nucleic acid sequences) over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity. A sequence that is identical at every position in comparison to a reference sequence is said to be 100% identical to the reference sequence, and vice-versa.

Polynucleotide sequences may have substantial identity, substantial homology, or substantial complementarity to the selected region of the target gene. As used herein “substantial identity” and “substantial homology” indicate sequences that have sequence identity or homology to each other. Generally, sequences that are substantially identical or substantially homologous will have about 75%, 80%, 85%, 90%, 91%, 92%, 93%, 94%, 95%, 96%, 97%, 98%, 99%, or 100% sequence identity wherein the percent sequence identity is based on the entire sequence and is determined by GAP alignment ixsing default parameters (GCG, GAP version 10, Accelrys, San Diego, Calif.). GAP uses the algorithm of Needleman and Wunsch ((1970) J Mol Biol 48:443-453) to find the alignment of two complete sequences that maximizes the number of matches and minimizes the number of sequence gaps. Sequences which have 100% identity are identical. “Substantial complementarity” refers to sequences that are complementary to each other, and are able to base pair with each other. In describing complementary sequences, if all the nucleotides in the first sequence will base pair to the second sequence, these sequences are fully complementary.

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10. The National Center for Biotechnology Information (NCBI) Basic Local Alignment Search Tool (BLAST™; Altschul et al. (1990)) is available from several sources, including the National Center for Biotechnology Information (Bethesda, Md.), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default BLOSUM62 matrix set to default parameters. Nucleic acid sequences with even greater similarity to the reference sequences will show increasing percentage identity when assessed by this method.

As used herein, the term “homologous” with regard to a contiguous nucleic acid sequence, refers to contiguous nucleotide sequences that hybridize under appropriate conditions to the reference nucleic acid sequence. For example, homologous sequences may have from about 70%-100, or more generally 80% to 100% sequence identity, such as about 81%; about 82%; about 83%; about 84%; about 85%; about 86%; about 87%; about 88%; about 89%; about 90%; about 91%; about 92%; about 93%; about 94% about 95%; about 96%; about 97%; about 98%; about 98.5%; about 99%; about 99.5%; and about 100%. The property of substantial homology is closely related to specific hybridization. For example, a nucleic acid molecule is specifically hybridizable when there is a sufficient degree of complementarity to avoid non-specific binding of the nucleic acid to non-target sequences under conditions where specific binding is desired, for example, under stringent hybridization conditions.

Homologs, variants and alleles of the target molecules or proteins of the invention can be identified by conventional techniques. As used herein, a homolog or variant to a polypeptide is a polypeptide from a plant source that has a high degree of structural similarity to the identified polypeptide.

The term, “operably linked,” when used in reference to a regulatory sequence and a coding sequence, means that the regulatory sequence affects the expression of the linked coding sequence. “Regulatory sequences,” or “control elements,” refer to nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; polyadenylation recognition sequences; etc. Particular regulatory sequences may be located upstream and/or downstream of a coding sequence operably linked thereto. Also, particular regulatory sequences operably linked to a coding sequence may be located on the associated complementary strand of a double-stranded nucleic acid molecule.

As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a nucleotide sequence encoding a signal sequence which may be operably linked to a coding sequence for expression in a cell. A “plant promoter” may be a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred.” Promoters which initiate transcription only in certain tissues are referred to as “tissue-specific.” A “cell type-specific” promoter primarily drives expression in certain cell types in one or more organs, for example, vascular cells in roots or leaves. An “inducible” promoter may be a promoter which may be under environmental control. Examples of environmental conditions that may initiate transcription by inducible promoters include anaerobic conditions and the presence of light. Tissue-specific, tissue-preferred, cell type specific, and inducible promoters constitute the class of “non-constitutive” promoters. A “constitutive” promoter is a promoter which may be active under most environmental conditions or in most cell or tissue types.

As used herein, the term “transformation” or “genetically modified” refers to the transfer of one or more nucleic acid molecule(s) into a cell. A microorganism is “transformed” or “genetically modified” by a nucleic acid molecule transduced into the bacteria when the nucleic acid molecule becomes stably replicated by the bacteria. As used herein, the term “transformation” or “genetically modified” encompasses all techniques by which a nucleic acid molecule can be introduced into such a bacteria.

The term “vector” refers to some means by which DNA, RNA, a protein, or polypeptide can be introduced into a host. The polynucleotides, protein, and polypeptide which are to be introduced into a host can be therapeutic or prophylactic in nature; can encode or be an antigen; can be regulatory in nature, etc. There are various types of vectors including virus, plasmid, bacteriophages, cosmids, and bacteria.

An “expression vector” is nucleic acid capable of replicating in a selected host cell or organism. An expression vector can replicate as an autonomous structure, or alternatively can integrate, in whole or in part, into the host cell chromosomes or the nucleic acids of an organelle, or it is used as a shuttle for delivering foreign DNA to cells, and thus replicate along with the host cell genome. Thus, an expression vector are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette.” In contrast, as described in the examples herein, a “cassette” is a polynucleotide containing a section of an expression vector of this invention. The use of the cassettes assists in the assembly of the expression vectors. An expression vector is a replicon, such as plasmid, phage, virus, chimeric virus, or cosmid, and which contains the desired polynucleotide sequence operably linked to the expression control sequence(s). A polynucleotide sequence is operably linked to an expression control sequence(s) (e.g., a promoter and, optionally, an enhancer) when the expression control sequence controls and regulates the transcription and/or translation of that polynucleotide sequence.

Unless otherwise indicated, a particular nucleic acid sequence also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). Because of the degeneracy of nucleic acid codons, one can use various different polynucleotides to encode identical polypeptides. As provided below, the table contains information about which nucleic acid codons encode which amino acids.

Amino Acid Nucleic Acid Codons

Nucleic Acid

| Amino Acid | Codons |
|---|---|
| Ala/A | GCT, GCC, GCA, |
| | GCG |
| Arg/R | CGT, CGC, CGA, |
| | CGG,AGA, AGG |
| Asn/N | AAT, AAC |
| Asp/D | GAT, GAC |
| Cys/C | TGT, TGC |
| Gln/Q | CAA, CAG |
| Glu/E | GAA, GAG |
| Gly/G | GGT, GGC, GGA, |
| | GGG |
| His/H | CAT, CAC |
| Ile/I | ATT, ATC, ATA |
| Leu/L | TTA, TTG, CTT, |
| | CTC, CTA, CTG |
| Lys/K | AAA, AAG |
| Met/M | ATG |
| Phe/F | TTT, TTC |
| Pro/P | CCT, CCC, CCA, |
| | CCG |
| Ser/S | TCT, TCC, TCA, |
| | TCG, AGT, AGC |
| Thr/T | ACT, ACC, ACA, |
| | ACG |
| Trp/W | TGG |
| Tyr/Y | TAT, TAC |
| Val/V | GTT, GTC, GTA, |
| | GTG |

In addition to the degenerate nature of the nucleotide codons which encode amino acids, alterations in a polynucleotide that result in the production of a chemically equivalent amino acid at a given site, but do not affect the functional properties of the encoded polypeptide, are well known in the art. “Conservative amino acid substitutions” are those substitutions that are predicted to interfere least with the properties of the reference polypeptide. In other words, conservative amino acid substitutions substantially conserve the structure and the function of the reference protein. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine or histidine, can also be expected to produce a functionally equivalent protein or polypeptide. As provided below, the table provides a list of exemplary conservative amino acid substitutions. Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

Amino Acids and Conservative Substitutes

| Amino Acid | Conservative Substitute |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, |
| | Glu |
| Ile | Leu, Val |
| Leu | Ile, Val |
| Lys | Arg, Gln, Glu |
| Met | Ile, Leu |
| Phe | His, Leu, Met, |
| | Trp, Tyr |
| Ser | Cys, Thr |
| Thr | Ser, Val |
| Trp | Phe, Tyr |
| Tyr | His, Phe, Trp |
| Val | Ile, Leu, Thr |

Oligonucleotides and polynucleotides that are not commercially available can be chemically synthesized e.g., according to the solid phase phosphoramidite triester method first described by Beaucage and Caruthers, Tetrahedron Letts. 22:1859-1862 (1981), or using an automated synthesizer, as described in Van Devanter et al., Nucleic Acids Res. 12:6159-6168 (1984). Other methods for synthesizing oligonucleotides and polynucleotides are known in the art. Purification of oligonucleotides is by either native acrylamide gel electrophoresis or by anion-exchange HPLC as described in Pearson & Reanier, J. Chrom. 255:137-149 (1983). Additional methods are known by those of ordinary skill in the art.

As used herein, the term “endogenous” refers to any material from or produced inside an organism, cell, tissue or system.

As used herein, the term “exogenous” refers to any material introduced from or produced outside an organism, cell, tissue or system.

The term “recombinant” when used with reference, e.g., to a cell, or nucleic acid, protein, or vector, indicates that the cell, organism, nucleic acid, protein or vector, has been modified by the introduction of a heterologous nucleic acid or protein, or the alteration of a native nucleic acid or protein, or that the cell is derived from a cell so modified. Thus, for example, recombinant cells may express genes that are not found within the native (nonrecombinant or wild-type) form of the cell or express native genes that are otherwise abnormally expressed-over-expressed, under expressed or not expressed at all.

The terms “transgenic”, “transformed”, “transformation”, and “transfection” are similar in meaning to “recombinant”. “Transformation”, “transgenic”, and “transfection” refer to the transfer of a polynucleotide into the genome of a host organism or into a cell. Such a transfer of polynucleotides can result in genetically stable inheritance of the polynucleotides or in the polynucleotides remaining extra-chromosomally (not integrated into the chromosome of the cell). Genetically stable inheritance may potentially require the transgenic organism or cell to be subjected for a period of time to one or more conditions which require the transcription of some or all of the transferred polynucleotide in order for the transgenic organism or cell to live and/or grow. Polynucleotides that are transformed into a cell but are not integrated into the host's chromosome remain as an expression vector within the cell. One may need to grow the cell under certain growth or environmental conditions in order for the expression vector to remain in the cell or the cell's progeny. Further, for expression to occur the organism or cell may need to be kept under certain conditions. Host organisms or cells containing the recombinant polynucleotide can be referred to as “transgenic” or “transformed” organisms or cells or simply as “transformants,” as well as recombinant organisms or cells.

A genetically altered organism is any organism with any change to its genetic material, whether in the nucleus or cytoplasm (organelle). As such, a genetically altered organism can be a recombinant or transformed organism. A genetically altered organism can also be an organism that was subjected to one or more mutagens or the progeny of an organism that was subjected to one or more mutagens and has changes in its DNA caused by the one or more mutagens, as compared to the wild-type organism (i.e, organism not subjected to the mutagens). Also, an organism that has been bred to incorporate a mutation into its genetic material is a genetically altered organism. For the purposes of this invention, the organism is a plant.

The term “plant” includes whole plants, plant organs, progeny of whole plants or plant organs, embryos, somatic embryos, embryo-like structures, protocorms, protocorm-like bodies (PLBs), and suspensions of plant cells. Plant organs comprise, e.g., shoot vegetative organs/structures (e.g., leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, trichomes and the like). The class of plants that can be used in the method of the invention is generally as broad as the class of higher and lower plants amenable to the molecular biology and plant breeding techniques described herein, specifically angiosperms (monocotyledonous (monocots) and dicotyledonous (dicots) plants including eudicots. It includes plants of a variety of ploidy levels, including aneuploid, polyploid, diploid, haploid and hemizygous. In one preferred embodiment, the genetically altered plants described herein can be dicot crops, such as citrus.

The terms “approximately” and “about” refer to a quantity, level, value or amount that varies by as much as 30%, or in another embodiment by as much as 20%, and in a third embodiment by as much as 10% to a reference quantity, level, value or amount. As used herein the term “increased, or decreased with respect to the use or effect of an antimicrobial peptide means increased, or decreased compared to wild-type.

Additionally, the term low, for example when describing low toxicity means that the levels of toxicity of the antimicrobial peptide would be approximately the same as a application of a single amphipathic helical peptide or that the levels of toxicity as at a level that a mammalian cell or host will not exhibit a significantly toxic effect. In some embodiments, the novel antimicrobial peptides may exhibits low or no toxicity, which may mean that they exhibit similar levels of toxicity or phytotoxcicity as compared to an endogenous amphipathic peptide.

Additionally, the term low, for example when describing low phytotoxcicity means that the levels of toxicity of the antimicrobial peptide would be approximately the same as a application of a single amphipathic helical peptide or that the levels of toxicity as at a level that the plants growth and other properties an functions may not be significantly affected by a antimicrobial peptide.

As used herein, the singular form “a”, “an”, and “the” include plural references unless the context clearly dictates otherwise. For example, the term “a peptide” includes both a single peptide and a plurality of peptides.

As noted above, the compositions and substances set forth above can be used to modulate the amount of Candidatus Liberibacter spp. infestation in plants, their seeds, roots, fruits, foliage, stems, tubers, and in particular, inhibit and/or prevent Candidatus Liberibacter spp. infection, in particular, decrease the rate and/or degree of spread of Candidatus Liberibacter spp. infection in plants. While a preferred embodiment may include citrus plants, additional plants, include but are not limited to fruits (e.g., strawberry, blueberry, blackberry, peach and other stone fruits), vegetable (e.g., tomato, squash, pepper, eggplant, potatoes, carrots), or grain crops (e.g., soy, wheat, rice, corn, sorghum), trees, flowers, ornamental plants, shrubs (e.g., cotton, roses), bulb plants (e.g., onion, garlic) or vines (e.g., grape vine), turf, tubers (e.g. potato, carrots, beets). Alternatively, the inventive compositions can be used to modulate the amount of Candidatus Liberibacter spp. infection in plants and in particular, prevent or inhibit Candidatus Liberibacter spp. infection and/or decrease the rate and/or degree of spread of disease infection in said plants.

Persons of skill are aware of various methods to apply microbial-based compositions, to plants for surface application or for uptake, and any of these methods are contemplated for use in this disclosure. Methods of administration to plants include, by way of non-limiting example, application to any part of the plant, by inclusion in irrigation water, by injection into the plant or into the soil surrounding the plant, by exposure of the root system to aqueous solutions containing the compounds, by use in hydroponic or aeroponic systems, by culture of individual or groups of plant cells in media containing the inducer, by seed treatment, by exposure of cuttings of citrus plants used for grafting to aqueous solutions containing the compounds, by application to the roots, stems or leaves, or by application to the plant interior, or any part of the plant to be treated. Any means known to those of skill in the art is contemplated. One mode of administration includes those where the compositions are applied at, on or near the roots of the plant, or trunk injection. Application of microbial-based compositions can be performed in a nursery setting, a greenhouse, hydroponics facility, or in the field, or any setting where it is desirable to treat plants to prevent the likelihood of disease, or to treat disease and its effects, for example in plants which have been or can become exposed to HLB or Clas infection. The methods and compounds of this disclosure can be used to treat infection with any Candidatus Liberibacter species or type and can be used to improve plant defenses in plants which are not infected. Thus, any plant in need, in the context of this disclosure, includes any and all plants for which improvements in health and vigor, growth and productivity or ability to combat disease is desired. Citrus or other plants susceptible to diseases such as HLB or infection by Candidatus Liberibacter species, whether currently infected or in potential danger of infection.

As defined herein, with respect to any amphipathic helical peptide or antimicrobial peptide the terms “derived from” or “from” means directly isolated or obtained from a particular source or alternatively having identifying characteristics of a substance or organism isolated or obtained from a particular source. In the event that the “source” is an organism, “derived from” or “from” means that it may be isolated or obtained from the organism itself or from the medium used to culture or grow said organism.

The term “citrus”, as used herein, refers to any plant of the genus Citrus, family Rutaceae, and includes Citrus maxima (Pomelo), Citrus medica (Citron), Citrus micrantha (Papeda), Citrus reticulata (Mandarin orange), Citrus trifolata (trifoliate orange), Citrus japonica (kumquat), Citrus australasica (Australian Finger Lime), Citrus australis (Australian Round lime), Citrus glauca (Australian Desert Lime), Citrus garrawayae (Mount White Lime), Citrus gracilis (Kakadu Lime or Humpty Doo Lime), Citrus inodora (Russel River Lime), Citrus warburgiana (New Guinea Wild Lime), Citrus wintersii (Brown River Finger Lime), Citrus halimii (limau kadangsa, limau kedut kera); Citrus indica (Indian wild orange), Citrus macroptera, and Citrus latipes. Hybrids also are included in this definition, for example Citrus.times.aurantiifolia (Key lime), Citrus, times, aurantium (Bitter orange), Citrus. times. latifolia (Persian lime), Citrus.times.limon (Lemon), Citrus. times. limonia (Rangpur), Citrus. times. paradisi (Grapefruit), Citrus.times. sinensis (Sweet orange), Citrus.times.tangerina (Tangerine), Poncirus trifoliata.times.C. sinensis (Carrizo citrange), and any other known species or hybrid of genus Citrus. Citrus known by their common names include, Imperial lemon, tangelo, orangelo, tangor, kinnow, kiyomi, Minneola tangelo, oroblanco, sweet orange, ugli, Buddha's hand, citron, lemon, orange, bergamot orange, bitter orange, blood orange, calamondin, Clementine, grapefruit, Meyer lemon, Rangpur, tangerine, and yuzu, and these also are included in the definition of citrus or Citrus.

The term treatment and/or prevention means providing an “effective amount” or “therapeutically effective amount,” which means any amount of the compound or composition which serves its purpose, for example, treating plant disease, improving the ability of plants to defend against disease, reducing disease symptoms, treating HLB, increasing resistance to HLB, minimizing crop yield decreases due to plant disease, improving crop productivity, and increasing crop quality.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “any combination thereof” as used herein refers to all permutations and combinations of the listed items preceding the term. For example, “A, B, C, or any combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

The present invention is further illustrated in the following examples, which should not be taken to limit the scope of the invention.

### EXAMPLES

### Example 1

### Evolution and Characterization of Bacterial Resistance Against a Host Amphipathic Helix

As noted above, the biggest drawback of the host amphipathic helical peptides is the evolution of bacterial resistance, i.e., ability of the bacteria to block attachment, insertion, and rupture of the bacterial membrane by the peptides. The most direct way to determine how the resistant strain blocks the activity of a given host amphipathic helical peptide is to first generate a resistant strain against the peptide, then sequence the genome of the resistant strain and finally, identify the mutated genes that adversely affect attachment, insertion, and rupture of the bacterial membrane by the peptide. However, these experiments cannot be done with Liberibacter since it is not culturable. Therefore, the present inventors selected two human E. coli strains to develop resistance against an endogenous amphipathic helical peptide and to identify the mutated genes that confer resistance by blocking attachment, insertion, and rupture of the bacterial membrane by the peptide. Tables 1 and 2 below list the mutated genes in the target E. coli strains resistant to an endogenous citrus amphipathic helical peptide, P11 with sequence KKLIKKILKIL wherein basic and hydrophobic amino acid patches alternate in the structure. As shown by the present inventors, all of the mutations led to disabling the functions of the genes. Also, mutations in multiple genes (and not a single one or a few) are required to confer resistant. As also shown, except for two target genes, the rest of the mutations occur in different genes in the two different resistant strains. Thus, different pathways involving different gene mutations may lead to bacterial resistance against the same antimicrobial peptide, P11. Nonetheless, the two different pathways of gene mutations in two different E. coli strains appear to hinder attachment, insertion, or rupture of the bacterial membrane by P11 to confer resistance.

As highlighted in Tables 1 and 2 below, the insertion mutations in the rsxC and mlaD genes are common in both the resistant E. coli strains. The insertions in these two genes lead to disabling of their functions. The MlaD protein is involved in transferring phospholipid from the outer-membrane to the inner-membrane. Thus, the loss of MlaD function may result in a thicker outer-membrane thereby hindering the insertion of the endogenous amphipathic helix. Disabling insertion mutations in waaP/rfap and yejP in the resistant E. coli BL21 strain decrease the attachment of P11 to the outer-membrane of the bacterium. waaP/rfap mutation causes removal of phosphate groups on mid/outer region of LPS whereas yejP mutation leads to removal of phosphatidyl-ethanolamine from lipid A, both of which reduce the negative charge on the outer-membrane and therefore, P11 attachment. Disabling insertion mutation in asmA in the resistant E. coli BL:21 strain leads to defective organization OmpF porin on the outer-membranes leading to decrease in P11 attachment. Intergenic insertion mutation in leuO and leucine leader peptide genes causes disruption in the leuO operator and suppression of the expression of the leuO gene. This leads to glycylination of lipid A and decrease in P11 attachment. A disabling deletion mutation in the hemagglutinin fhaC gene and insertion mutation in the phospholipase pldA gene in the resistant E. coli ATCC25922 strain decreases P11 attachment whereas the disabling insertion mutation in the entS/ybdA transporter gene decreases P11 insertion. Intergenic insertion in the wcaK and wzxC genes in the resistant E. coli ATCC25922 strain causes suppression of extracellular polysaccharide colonic acid leading to decrease in P11 attachment.

In summary, the present inventors have demonstrated that resistance in E. coli due to an endogenous amphipathic helix, P11. As a result, the present inventors have demonstrated that: (i) multiple gene and intergenic mutations may be needed for resistance against antimicrobial peptides (AMP) via a decrease in attachment and insertion; (ii) different sets of mutations may lead to decrease in attachment and insertion and thus in antimicrobial peptide (e.g., P11) activity; (iii) mutations in multiple genes contribute to the decrease in antimicrobial peptide (e.g., P11) attachment and/or insertion; and (iv) only a subset of the genes that confer resistance to anti-microbial peptides (e.g., P11) is also present in CLas; therefore, mutations on this subset and probably additional genes may be needed for CLas resistance against the same an antimicrobial peptide.

### Example 2

### Design of Next-Generation Host-Derived Amphipathic Helical Antimicrobial Peptides

In one preferred embodiment, the present inventors demonstrate that the design of novel amphipathic helix based upon host analogs may lead to anti-CLas therapeutics that are more active and less toxic than the host analogs and not expected to be susceptible to bacterial resistance. As noted above, the host single amphipathic helix causes structural instability resulting in decreased activity as well as susceptibility to bacterial/host proteases. The present inventors sought to generate a novel AMP by coupling two amphipathic helices, in this case P11 through a linker domain to generate a helix-turn-helix AMP. Specifically, as shown in FIG. 1, the present inventors joined two host helices by a GPGR-turn or linker. In this configuration, the GPGR-turn or linker may block end-fraying at the N-terminus of one helix and C-terminus of the other helix. This configuration leads to a helix-turn-helix scaffold, which exhibits higher stability than the host single amphipathic helix and therefore, less susceptible to protease digestion. In addition, as shown in FIG. 1, hydrophobic residues are in the interior of the scaffold whereas the basic residues are on the surface. Such an arrangement of the hydrophobic and basic residues may facilitate efficient CLas membrane attachment and insertion of the helix-turn-helix scaffold, such helix-turn-helix configuration being generally represented as P26. The stability of the P26 helix-turn helix peptide can be further improved by replacement of two hydrophobic residues (one from each helix) by two cysteines forming a S-S bridge, identified generally as cysP26 (see FIG. 1). Finally, end-fraying of the helices can be further blocked in a cyclic scaffold where two helices and two GPGR-turns may be coupled together, identified generally as cycP30 (see FIG. 1).

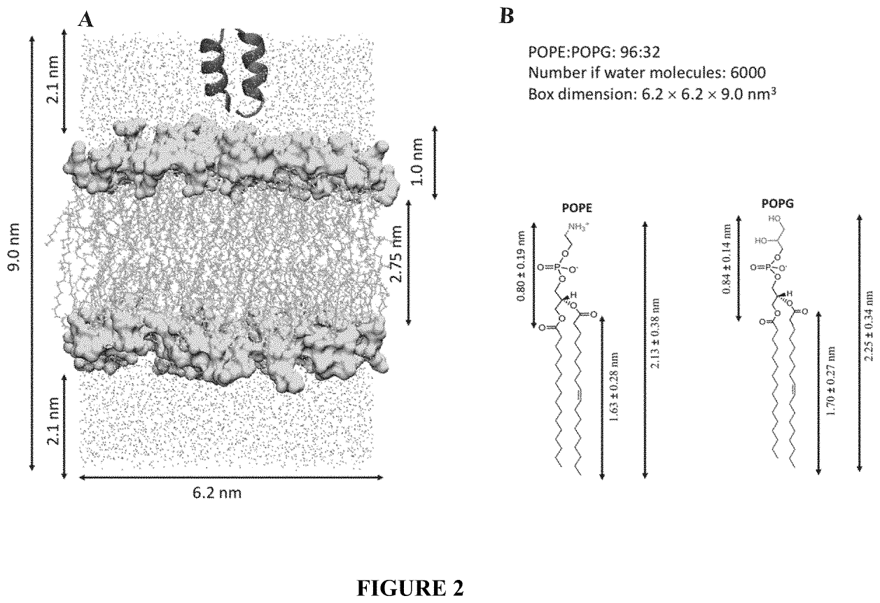

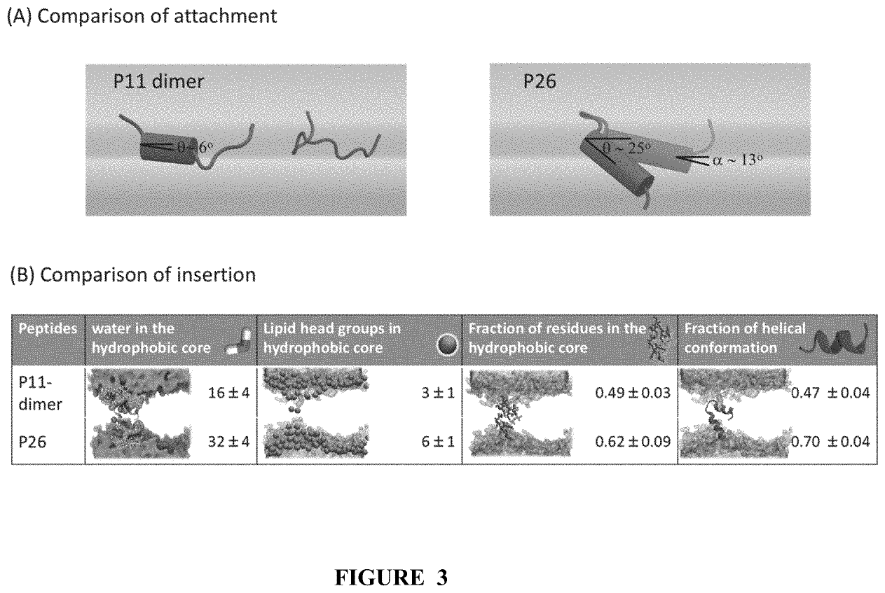

The present inventors net performed structure-activity analysis of the host single helix, P11, and the engineered helix-turn-helix scaffold, P26. For this, we performed molecular dynamics (MD) simulation of P11 and P26 in synthetic lipid bilayer, or in other word a simulated bacterial membrane. FIG. 2 describes the composition and the dimensions of the water:lipid bilayer. First, a MD simulation was performed using GROMOS force-field to monitor the attachment of a P11 dimer and P26 from water to the lipid. Average attachment profiles of P11-dimer and P26 were calculated from the 1 μsec trajectory after 100 ns of equilibration. FIG. 3A shows the difference in membrane attachment of P11-dimer and P26. Notably, the P26 positions deeper into the lipid bilayer than P11-dimer while still retaining the helical conformation whereas P11-dimer undergoes helix to coil transition.

MD simulation of P11 dimer and P26 inside the lipid core may also be informative of the inventive technology. Specifically, four parameters are computed from 1 μsec MD trajectory after 150 nsec of equilibration. These are: number of water molecules in the lipid core; number of polar lipid head groups flipped into the lipid core; fraction of residues in the hydrophobic core; and helical content. The present inventors predicted that the higher the values of these parameters for a peptide, the higher the insertion efficiency and thus the bactericidal activity. On these criteria, P26 is expected to show higher bactericidal activity than P11-dimer. The present inventors tested the prediction from the MD simulation studies by measuring the minimum inhibitory concentrations (MIC) of P11 and P26 against three E. coli wildtype and P11-resistant strains. Table 3 below lists the MIC values, which show not only is P26 more active than P11 but also that it is not susceptible to bacterial resistance.

Further structure-activity analysis of several helix-turn-helix P26 scaffolds based upon citrus P11 has been performed (see Table IV). These include P26, cysP26, and cycP30 shown in FIG. 1. In addition, the present inventors initiated structure-activity studies on the helix-turn-helix 28P scaffolds based upon citrus single 12 amino acid peptide 12P (see Table 4). Finally, structural analysis of helix-turn-helix scaffolds based upon single amphipathic citrus helices of length 10 to 30 amino acids is generally shown below in Table 4, which specifically IV show helix-turn-helix scaffolds based upon citrus single helices 10P and 27P.

### Example 3

### Effect of Different Peptides on the Viability of N. benthamiana Mmesophyll Protoplasts

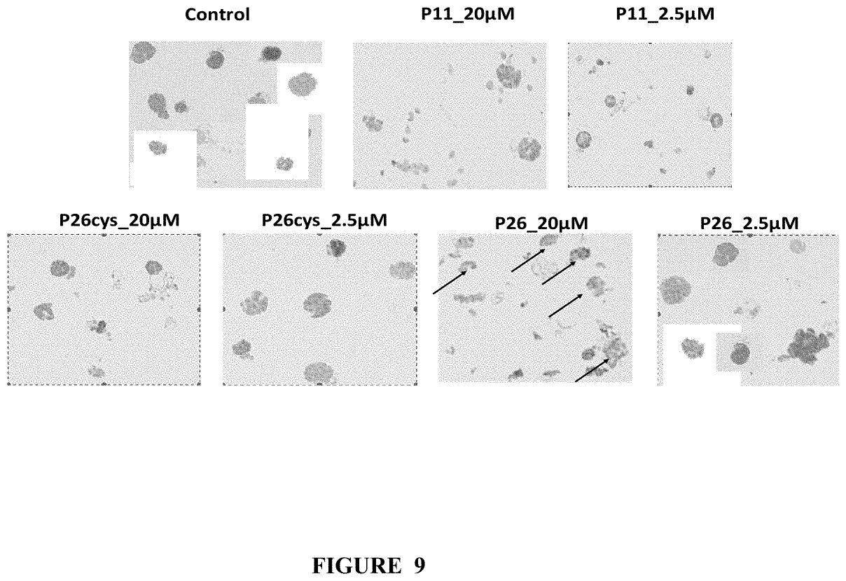

As shown in FIG. 9, young leaves were taken and incubated for 5 h in dark with cellulase and macerozyme in buffer containing mannitol, calcium chloride and MES. Protoplasts were released by passing through cheese cloth. Protoplasts were incubated with different peptide concentrations and photographs were taken after 1 h. Cells with spherical shape are defined as viable. Hints to cell death include loss of spherical shape, release of chloroplast and protoplast aggregation. The arrows here indicate cells that have been damaged due to the toxic effect of the peptides. P11 is the single helix from which P26 and cysP26 are engineered.

### Example 4

### Efficacy Testing Assays of Novel Helix-Turn-Helix Scaffolds

Two bactericidal assays were performed, which involve treatment of the host single helix (P11 or P11-R) and designed helix-turn-helix scaffolds on CLas infected citrus leaves and psyllids and subsequent clearance of CLas. Leaf and psyllid assays are described below:

Citrus Leaf Disk Assay

CLas-infected leaves were washed in mild soap. A 4mm biopsy punch was used to remove small disks from the midrib of the leaf. Disks were arranged in groups on a 96-well plate with each leaf having a disk in each control or treatment solution. 200 μL of sterilized tap water with 100 μM potassium phosphate buffer (pH 7.0) was used as incubation buffer and for negative controls. Antimicrobial peptides were added to buffer at 0.5 mM and incubated for 48 hours with leaf disks, which were removed and individually processed using liquid nitrogen grinding and phenol pH 8.0 for total nucleic acid extraction.

DNA was isolated using DNeasy plant mini kit (Qiagen, Gaithersburg, Md., USA), and the quantity and quality were determined by a NanoDrop Spectrophotometer (Thermo Scientific, Wilmington, Del., USA). Real-time qPCR measurements were made using Gotaq RT-OneStep and Las Long primers with 10Ong of nucleic acid loaded for each reaction using an ABI7500 thermal cycler (Applied Biosystems, Foster City, Calif., USA). The threshold cycle (Cₜ) values were used to calculate bacterial titer using the standard curve method (Shi et al., 2017). CLas titer [log (las copy number)] before treatment (μ) and after treatment (f) were determined using Las long primers by qPCR. The Las clearance percentage was calculated according to the following equation: % clearance=[1−f/μ]×100.

Psyllid Assay:

This assay involved the following steps: CLas isolation centrifugation and glycerol extraction, addition of (P11 or P11-R) and designed helix-turn-helix scaffolds, removal of psyllid DNA by PMAxx, and extraction and monitoring reduction of CLas DNA. CLas clearance were estimated by the method described in leaf disk assay.

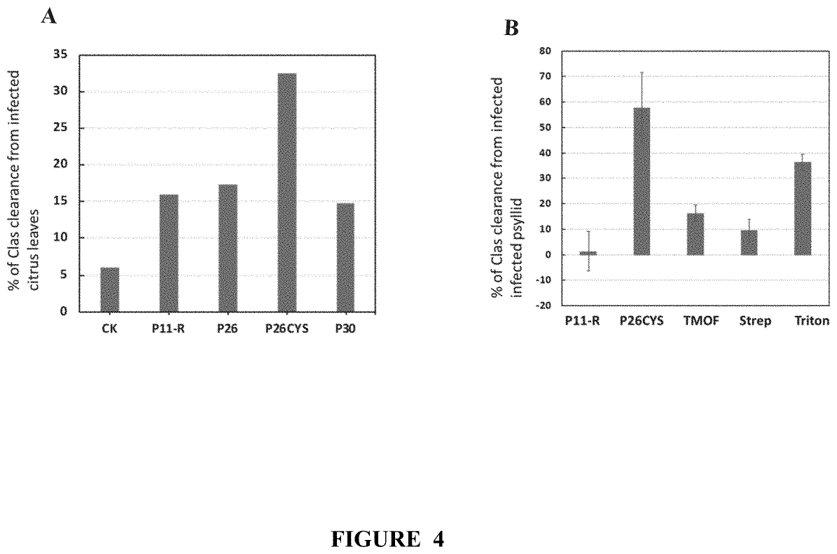

As shown in FIG. 4, the engineered helix-turn-helix scaffolds demonstrated the ability to clear CLas from infected citrus leaves and psyllids.

### Example 5

### Hemolytic Assay Analysis of Various Engineered Antimicrobial Peptides

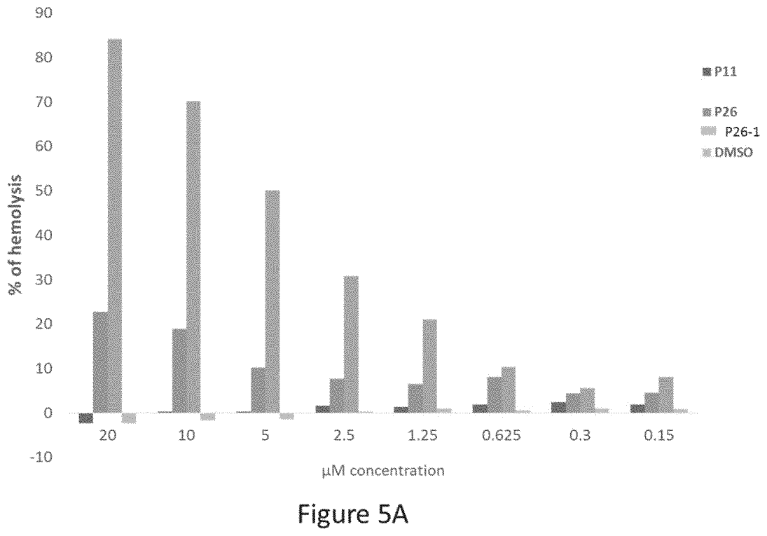

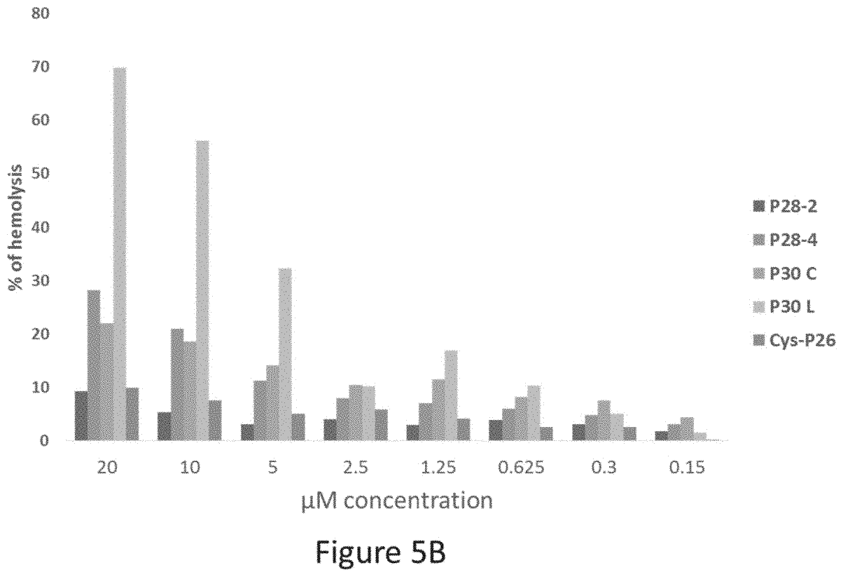

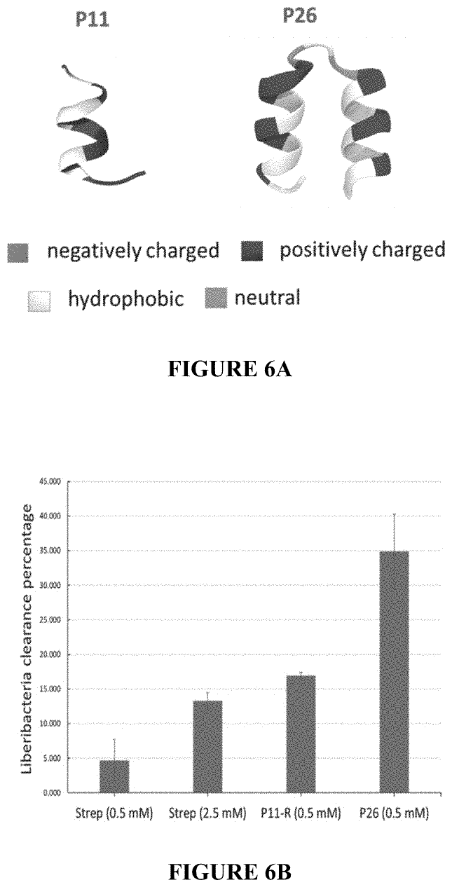

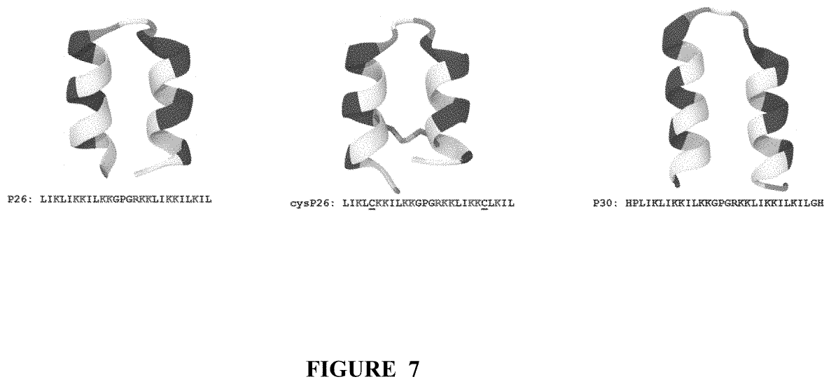

As show in in FIGS. 5 A and B, hemolytic assay was performed using the protocol described in Evans et al. (Evans B C, Nelson C E, Yu S S, Beavers K R, Kim A J, Li H, Nelson H M, Giorgio T D, Duvall C L. J Vis Exp. 2013 Mar. 9;(73):e50166. doi: 10.3791/50166). A 10% (v/v) suspension of human erythrocytes in PBS was stored at 4° C. When needed, the suspension was diluted 1:10 in PBS and 100 μl was added in triplicate to 100 μl of a 2-fold serial dilution series of peptide in a 96-well plate. Total hemolysis was achieved with 1% Tween 20. RBC with only PBS was used as a control. The plates were incubated at 37° C. for 1 h and centrifuged for 10 min at 3,000 rpm (900×g). Then, 160 μl of the supernatant was transferred to a new 96-well plate to measure the absorbance at 405 nm by using a microplate reader, and the percent hemolysis was calculated.

### Tables

TABLE 1
Mutations in genes and intergenic regions in P11-resistant E. coli BL21 genome.

| | | | | Effect of | CLas |
|---|---|---|---|---|---|
| Locus_tag | Gene name^{a} | Function | Mutation | mutation | homolog |
| Gene ORF | | | | | |
| ECBD_1518 | asmA | Assembly of outer membrane proteins | Insertion (1325 bp) | insertion | Yes |
| ECBD_1591 | waaP/rfap | LPS core biosynthesis | Insertion (2668 bp) | attachment | No |
| ECBD_0096 | rsxC | Electron transport complex subunit | Insertion (99 bp) | insertion | No |
| ECBD_2015 | yejM | inner membrane sulfatase | Insertion (1203 bp) | attachment | No |
| ECBD_2141 | mlaD | Phospholipid binding and transport | Deletion (11 bp) | insertion | No |
| ECBD_0549 | dusC | Catalyzes the synthesis of 5,6- | Insertion (1342 bp) | not known | Yes |
| | | dihydrouridine | | | |
| Intergenic regions | | | | | |
| ECBD_1425 | nrdB | Catalyzes the conversion of nucleotides | Insertion (110 bp) | growth and | Yes |
| ECBD_1426 | nrdA | to deoxynucleotides | | adaptation | Yes |
| ECBD_3540 | leuO | A global transcription factor | Insertion (1342 bp) | attachment | Yes |
| ECBD_3541 | leu operon leader | Involved in control of the biosynthesis | | | No |
| | peptide | of leucine | | | |
| ECBD_3641 | yjjW | glycine radical enzyme activase | Insertion (220 bp) | not known | No |
| ECBD_3642 | yjjV | Metal-dependent hydrolase | | | Yes |

Table 1 Legend:
asmA: Encodes an inner-membrane protein in LPS biogenesis and in outer-membrane protein organization which may facilitate antimicrobial peptide (AMP) entry.
waaP/rfaP: Encodes a kinase that phosphorylates heptose I in the E. coli LPS inner core.
asmA: Encodes an inner-membrane protein and is involved in the organization of an outer-membrane porin OmpF.
rsxC: Encodes a reductase that reduces and inactivates; the transcription factor SoxR is active only in the oxidized state and it turns on the transcription activator of superoxide induced genes such sodA and micF as well as a battery of genes that increases the susceptibility of E. coli to AMPs.
yejM: Encodes an inner-membrane sulfatase/phosphatase that transfers negatively charged phosphatidyl-ethanolamine from the inner- to the outer-membrane.
malD: Encodes an inner-membrane hexamer MlaD which complexes with (MlaE-MlaF-MlaB) dimer; MlaC transports phospholipid from the outer-member to the MlaEFBD complex; the phospholipid then becomes part of the inner membrane.
nrdAB: Encodes ribonucleotide reductases A and B; suppression of nrdB operator is compensated by higher activation of nrdA.
leuO: Encodes LeuO that directly represses carRS by binding to its promoter resulting in decreased expression of almEFG, which reduces lipid A glycinylation and increases susceptibility to AMPs.

TABLE 2
Mutations in genes and intergenic regions in P11-resistant E. coli ATCC 25922 genome.

| | Gene | | | Effect of | CLas |
|---|---|---|---|---|---|
| Locus_tag | name | Product | Mutation | mutation | homolog |
| Gene ORF | | | | | |
| DR76_3209 | rsxC | Electron transport complex subunit | Insertion (96 bp) | attachment | No |
| DR76_3439 | entS/ybdA | EntS/YbdA MFS transporter | Insertion (441 bp) | insertion | No |
| DR76_3969 | fhaC like | Hemagluttinin | Deletion (4 bp) | attachment | No |
| DR76_1224 | | tRNA-Glu | Insertion (23 bp) | to be determined | Yes |
| DR76_649 | pldA | phospholipase A | Insertion (1240 bp) | attachment | No |
| DR76_1305 | mlaD | Outer membrane lipid asymmetry | Deletion (9 bp) | | No |
| | | maintenance protein | | | |
| Intergenic regions | | | | | |
| DR76_2138 | gltP | Glutamate/aspartate:proton symporter | Insertion (213 bp) | possibly insertion | Yes |
| DR76_2139 | yjcO | Sel1 repeat family protein | | | No |
| DR76_2232 | | tRNA-Gly | Insertion (110 bp) | | Yes |
| DR76_2233 | | tRNA-Gly | | | Yes |
| DR76_1225 | | 23S ribosomal RNA | Insertion (233 bp) | growth | Yes |
| DR76_1226 | | 5S ribosomal RNA | | | Yes |
| DR76_4791 | wcaK | colanic acid biosynthesis pyruvyl | Insertion (191 bp) | attachment | No |
| | | transferase | | | |
| DR76_4792 | wzxC | Colanic acid inner-membrane transporter | | | No |
| DR76_2803 | clbR | LuxR family transcriptional regulator | Deletion (32 bp) | to be determined | Yes |
| DR76_2804 | clbD | Colibactin hybrid non-ribosomal | | | Yes |
| | | peptide synthetase | | | |

Table 2 Legend:
rsxC & mlaD: see the legend of Table I above.
entS/ybdA: Encodes an inner-membrane protein involved in enterobactin transport.
flaC like gene: Encodes a filamentous outer-membrane hemagglutinin.
pldA: Encodes an outer-membrane phospholipase.
gltP: Encodes an inner-membrane glutamate/aspartate transporter with 10 trans-membrane helices.
yjcO: Encodes a secreted helix-rich solenoid protein.
wcaK: Encodes pyruvyl D transferase in the colanic acid synthesis pathway.
wzxC: Encodes inner-membrane colonic acid transport protein.
clbR: Encodes regulator of colibactin (a genotoxic agent) synthesis.
clbB: Encodes a colibactin synthesis gene.

TABLE 3
Bactericidal activities of host amphipathic helix P11 and
engineered P26 on wildtype and P11-resistant E. coli strains.

| | MIC (μM) | |
|---|---|---|
| E. Coli Strains | P11 | P26 |
| ATCC-WT | 14.9 ± 2.0 | 1.65 ± 0.3 |
| ATCC-R | 192 ± 8.3 | 9.5 ± 0.7 |
| K12-WT | 4 ± 0.7 | 1.65 ± 0.3 |
| K12-R | 75 ± 11.0 | 4.0 ± 0.6 |
| BL21-WT | 17.7 ± 2.7 | 1.95 ± 0.3 |
| BL21-R | 194 ± 12.5 | 4.4 ± 0.3 |

TABLE 4
Endogenous citrus host amphipathic helix and the engineered helix-turn-helix
scaffolds.

| Endogenous | | |
|---|---|---|
| Helix | Engineered Helix-turn-Helix | Source |
| LIKLIKKILKK | LIKLIKKILKK-GPGR-KKLIKKILKIL | KDO64589 |
| (P11) | (P26) | (hypothetical |
| LIRLIRRILRR | LIRLIRRILRR-GPGR-RRLIRRILRIL | protein with |
| (11P1) | (26P1) | Armadillo/beta- |
| | LIRLLRRILRR-GPGR-RRLIRRLLRIL | catenin-like |
| | (26P2) | repeats; 11 aa |
| | LIRLLREILRR-GPGR-ERLIRRLLRIL | serves as a linker |
| | (26P3) | between the |
| | LIRLILRILRR-GPGR-RRLIRLILRIL | repeats) |
| | (26P4) | |
| | LIRLISRILRR-GPGR-RRLIRSILRIL | |
| | (26P5) | |
| | LIKLCKKILKK-GPGR-KKLIKKCLKIL | |
| | (cysP26) | |
| | LIKLIKKILKK-GPGR-KKLIKKILKIL-GPGR | |
| | (P30) | |
| | 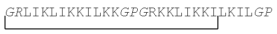 | |
| | (cycP30) | |
| | KKLIKKILKIL-GPGR-KKLIKEILKIL-GPGR- | |
| | KKLIKKILKIL | |
| | (41P) | |
| KRIVQRIKDF | KRIVQRIKDFLR-GPGR-KRIVQRIKDFLR | XP_006481400.1 |
| LR | (28P) | X (mitogen- |
| (12P) | KRLVQRLKDFLR-GPGR-KRLVQRLKDFLR | activated protein |
| KRLVQRLKD | (28P1) | kinase-binding |
| FLR | KRLIQRKRLIQR-GPGR-KRLIQRKRLIQR | protein 1 isoform |
| (12P1) | (28P1-2) | X1, Citrus |
| KRLIQRKRLI | | sinensis] |
| QR | | |
| (12P) | | |
| LYKKLSKKL | LYKKLSKKLL-GPGR-LYKKLSKKLL | KDO050283.1 |
| L (10P) | (24P) | (hypothetical |
| | | protein |
| | | CISIN_1g001207 |
| | | mg, Citrus |
| | | sinensis] |
| ALYLKDFKSS | ALYLKDFKSSKSLDVSALADLKHLKRL-GPGR- | XP_015390065.1 |
| KSLDVSALA | ALYLKDFKSSKSLDVSALADLKHLKRL | (disease |
| DLKHLKRL | (58P) | resistance protein |
| (27P) | | SUMM2-like, |
| | | Citrus sinensis) |

TABLE 5
Sequences of exemplary endogenous and engineered
antimicrobial peptides.

| | |
|---|---|
| P11 | KKLIKKILKIL |
| P26 | LIKLIKKILKKGPGRKKLIKKILKIL |
| P26-1 | LIRLIRRILRRGPGRRRLIRRILRIL |
| Cys-26-P | LIKLCKKILKKGPGRKKLIKKCLKIL |
| P28-2 | KRIVQRIKDFLRGPGRKRIVQRIKDFLR |
| P-30 C | GRLIKLIKKILKKGPGRKKLIKKILKILGP |
| P-30 L | HPLIKLIKKILKKGPGRKKLIKKILKILGH |
| P28-4 | KLIKLIKKILKKGPGRKKLIKKILKILK |

SEQUENCE LISTINGS

SEQ ID NO. 1
Amino Acid
Citrus sinensis
P11
LIKLIKKILKK
SEQ ID NO. 2
Amino Acid
Citrus sinensis
11P1
LIRLIRRILRR
SEQ ID NO. 3
Amino Acid
Artificial
P26
LIKLIKKILKKGPGRKKLIKKILKIL
SEQ ID NO. 4
Amino Acid
Artificial
26P1
LIRLIRRILRRGPGRRRLIRRILRIL
SEQ ID NO. 5
Amino Acid
Artificial
26P2
LIRLLRRILRR-GPGR-RRLIRRLLRIL
SEQ ID NO. 6
Amino Acid
Artificial
26P3
LIRLLREILRRGPGRERLIRRLLRIL
SEQ ID NO. 7
Amino Acid
Artificial
26P4
LIRLILRILRRGPGRRRLIRLILRIL
SEQ ID NO. 8
Amino Acid
Artificial
26P5
LIRLISRILRRGPGRRRLIRSILRIL
SEQ ID NO. 9
Amino Acid
Artificial
cysP26
LIKLCKKILKKGPGRKKLIKKCLKIL
SEQ ID NO. 10
Amino Acid
Artificial
P30
LIKLIKKILKKGPGRKKLIKKILKILGPGR
SEQ ID NO. 11
Amino Acid
Artificial
cycP30
GRLIKLIKKILKKGPGRKKLIKKILKILGP
SEQ ID NO. 12
Amino Acid
Artificial
41P
KKLIKKILKILGPGRKKLIKEILKILGPGRKKLIKKILKIL
SEQ ID NO. 13
Amino Acid
Citrus sinensis
12P
KRIVQRIKDFLR
SEQ ID NO. 14
Amino Acid
Citrus sinensis
12P1
KRLVQRLKDFLR
SEQ ID NO. 15
Amino Acid
Citrus sinensis
12P-2
KRLIQRKRLIQR
SEQ ID NO. 16
Amino Acid
Artificial
28P
KRIVQRIKDFLRGPGRKRIVQRIKDFLR
SEQ ID NO. 17
Amino Acid
Artificial
28P1
KRLVQRLKDFLRGPGRKRLVQRLKDFLR
SEQ ID NO. 18
Amino Acid
Artificial
28P1-2
KRLIQRKRLIQRGPGRKRLIQRKRLIQR
SEQ ID NO. 19
Amino Acid
Citrus sinensis
10P
LYKKLSKKLL
SEQ ID NO. 20
Amino Acid
Artificial
24P
LYKKLSKKLLGPGRLYKKLSKKLL
SEQ ID NO. 21
Amino Acid
Citrus sinensis
27P
ALYLKDFKSSKSLDVSALADLKHLKRL
SEQ ID NO. 22
Amino Acid
Artificial
58P
ALYLKDFKSSKSLDVSALADLKHLKRLGPGRALYLKDFKSSKSLDVSALA
DLKHLKRL
SEQ ID NO. 23
Amino Acid
Artificial
linker
GPGR

### REFERENCES

The following references are incorporated into the specification in their entirety:

1. https://www.freshfromflorida.com/Divisions-Offices/Plant-Industry/Agriculture-Industry/Citrus-Health-Response-Program/Citrus-Disease s/HLB-Citrus-Greening—references cited therein are incorporated by reference in their entirity.

2. http ://citrusindustry.net/2017/03/17/the-texas-psyllidhlb-experience/

3. http ://californiacitrusthreat.org/pest-disease

4. Blaustein R A, Lorca G L, Teplitski M. Challenges for Managing Candidatus Liberibacter spp. (Huanglongbing Disease Pathogen): Current Control Measures and Future Directions. Phytopathology. 2018 April;108(4):424-435. doi: 10.1094/PHYTO-07-17-0260-RVW. Epub 2018 Jan. 24.

5. file:///C:/Users/Goutam/Downloads/HLB FloridaEconomic%20Impact.pdf

6. http://www.syngenta-us.com/thrive/production/citrus-siege.html

7. J. Hu, J. Jiang, and N. Wang. Control of Citrus Huanglongbing via Trunk Injection of Plant Defense Activators and Antibiotics. Phytopathology, February 2018, Volume 108, Number 2, Pages 186-195.

8. Sundin G W, Wang N. Antibiotic Resistance in Plant-Pathogenic Bacteria. Annu Rev Phytopathol. 2018 Jun. 1. doi: 10.1146/annurev-phyto-080417-045946.

9. Sundin G W, Bender C L. Dissemination of the strA-strB streptomycin-resistance genes among commensal and pathogenic bacteria from humans, animals, and plants. Mol Ecol. 1996 Feb;5(1):133-43.

10. Thanner S, Drissner D, Walsh F. Antimicrobial Resistance in Agriculture. MBio. 2016 Apr. 19;7(2):e02227-15. doi: 10.1128/mBio.02227-15.

11. Dathe M, Wieprecht. Structural features of helical antimicrobial peptides: their potential to modulate activity on model membranes and biological cells Biochimica et Biophysica Acta 1462 (1999) 71-87.

12. Pushpanathan M, Gunasekaran P, Rajendhran J. Antimicrobial peptides: versatile biological properties. Int J Pept. 2013;2013:675391. doi: 10.1155/2013/675391. Epub 2013 Jun. 26.

13. Ganz T. The Role of Antimicrobial Peptides in Innate Immunity. INTEGR. COMP. BIOL., 43:300-304 (2003).

14. Yeung A T, Gellatly S L, Hancock R E. Multifunctional cationic host defence peptides and their clinical applications. Cell Mol Life Sci. 2011 Jul;68(13):2161-76. doi: 10.1007/s00018-011-0710-x. Epub 2011 May 15.

15. Taniguchi M, Ochiai A, Toyoda R, Sato T, Saitoh E, Kato T, Tanaka T. Effects of arginine and leucine substitutions on anti-endotoxic activities and mechanisms of action of cationic and amphipathic antimicrobial octadecapeptide from rice a-amylase. J Pept Sci. 2017 March;23(3):252-260. doi: 10.1002/psc.2983. Epub 2017 Feb 10.

16. Cruz J, Ortiz C, Guzman F, Fernandez-Lafuente R, Torres R. Antimicrobial peptides: promising compounds against pathogenic microorganisms. Curr Med Chem. 2014;21(20):2299-321.

17. Bell A. Antimalarial peptides: the long and the short of it. Curr Pharm Des. 2011;17(25):2719-31.

18. Joo H S, Fu C I, Otto M. Bacterial strategies of resistance to antimicrobial peptides.

Philos Trans R Soc Lond B Biol Sci. 2016 May 26;371(1695). pii: 20150292. doi: 10.1098/rstb.2015.0292.

19. Moravej H, Moravej Z, Yazdanparast M, Heiat M, Mirhosseini A, Moosazadeh Moghaddam M, Mirnej ad R. Antimicrobial Peptides: Features, Action, and Their Resistance Mechanisms in Bacteria. Microb Drug Resist. 2018 Jun. 29. doi: 10.1089/mdr.2017.0392.

20. Bahar A A, Ren D. Antimicrobial Peptides. Pharmaceuticals (Basel) 2013 December;6(12): 1543-1575. Published online 2013 Nov 28. Doi.

21. Yethon J A, Heinrichs D E, Mario A. Monteiro M A, Malcolm B. Perry M B, and Whitfield C. Involvement of waaY, waaQ, and waaP in the Modification of Escherichia coli Lipopolysaccharide and Their Role in the Formation of a Stable Outer Membrane. THE JOURNAL OF BIOLOGICAL CHEMISTRY Vol. 273, No. 41, Issue of October 9, pp. 26310- 26316, 1998.

22. Deng M, Misra R. Examination of AsmA and its effect on the assembly of Escherichia coli outer membrane proteins. Mol Microbiol. 1996 August;21(3):605-12.

23. Koo M S, Lee J H, Rah S Y, Yeo W S, Lee J W, Lee KL, Koh Y S, Kang S O, Roe J H. A reducing system of the superoxide sensor SoxR in Escherichia coli. EMBO J. 2003 Jun. 2;22(11):2614-22.

24. Warner D M, Levy S B. Different effects of transcriptional regulators MarA, SoxS and Rob on susceptibility of Escherichia coli to cationic antimicrobial peptides (CAMPs): Rob-dependent CAMP induction of the marRAB operon. Microbiology. 2010 February;156(Pt 2):570-8. doi: 10.1099/mic.0.033415-0. Epub 2009 Nov. 19.

25. Thong S, Ercan B, Torta F, Fong Z Y, Wong H Y, Wenk M R, Chng S S. Defining key roles for auxiliary proteins in an ABC transporter that maintains bacterial outer membrane lipid asymmetry. Elife. 2016 Aug. 16;5. pii: e19042. doi: 10.7554/eLife.19042.

26. Liang W, Deutscher M P. REP sequences: Mediators of the environmental stress response? RNA Biol. 2016;13(2):152-6. doi: 10.1080/15476286.2015.1112489. Epub 2015 Nov. 17.

27. Bina X R, Howard M F, Ante V M, Bina J E. Vibrio cholerae LeuO Links the ToxR Regulon to Expression of Lipid A Remodeling Genes. Infect Immun. 2016 Oct. 17;84(11):3161-3171. doi: 10.1128/IAI.00445-16.

28. Furrer J L, Sanders D N, Hook-Barnard I G, McIntosh M A. Export of the siderophore enterobactin in Escherichia coli: involvement of a 43 kDa membrane exporter. Mol Microbiol. 2002 June;44(5):1225-34.

29. Guerin J, Saint N, Baud C, Meli A C, Etienne E, Locht C, Vezin H, Jacob-Dubuisson F. Dynamic interplay of membrane-proximal POTRA domain and conserved loop L6 in Omp85 transporter FhaC. Mol Microbiol. 2015 October;98(3):490-501. doi: 10.1111/mmi.13137. Epub 2015 Aug. 14.

30. May K L, Silhavy T J. The Escherichia coli Phospholipase PldA Regulates Outer Membrane Homeostasis via Lipid Signaling. MBio. 2018 Mar. 20;9(2). pii: e00379-18. doi:

10.1128/mBio.00379-18.

31. Rahman M, Ismat F, Jiao L, Baldwin J M, Sharples D J, Baldwin S A, Patching S G. Characterisation of the DAACS Family Escherichia coli Glutamate/Aspartate-Proton Symporter GltP Using Computational, Chemical, Biochemical and Biophysical Methods. J Membr Biol. 2017 April;250(2):145-162. doi: 10.1007/s00232-016-9942-x. Epub 2016 Dec. 26.

32. https://www.uniprot.org/uniprot/POAF56

33. Ranjit D K, Young K D. Colanic Acid Intermediates Prevent De Novo Shape Recovery of Escherichia coli Spheroplasts, Calling into Question Biological Roles Previously Attributed to Colanic Acid. J Bacteriol. 2016 Mar. 31;198(8):1230-40. doi: 10.1128/JB.01034-15. Print 2016 Apr.

34. Putze J, Hennequin C, Nougayrede J P, Zhang W, Homburg S, Karch H, Bringer M A, Fayolle C, Carniel E, Rabsch W, Oelschlaeger T A, Oswald E, Forestier C, Hacker J, Dobrindt U. Genetic structure and distribution of the colibactin genomic island among members of the family Enterobacteriaceae. Infect Immun. 2009 November;77(11):4696-703. doi: 10.1128/IAI.00522-09. Epub 2009 Aug. 31.

35. Scholtz J M, Baldwin R L. The mechanism of alpha-helix formation by peptides. Annu Rev Biophys Biomol Struct. 1992;21:95-118.

36. Castelletto V, Barnes R H, Karatzas K A, Edwards-Gayle C J C, Greco F, Hamley I W, Rambo R, Seitsonen J, Ruokolainen J. Arginine-Containing Surfactant-Like Peptides: Interaction with Lipid Membranes and Antimicrobial Activity. Biomacromolecules. 2018 May 16. doi: 10.1021/acs.biomac.8b00391.

37. Senac C, Urbach W, Kurtisovski E, HUnenberger P H, Horta BA C, Taulier N, Fuchs P F J. Simulating Bilayers of Nonionic Surfactants with the GROMOS-Compatible 2016H66 Force Field. Langmuir. 2017 Oct. 3 ;33 (39): 10225-10238. doi: 10.1021/acs.langmuir.7b01348. Epub 2017 Sep. 20.

What is claimed is:

1-174. (canceled)

175. An antimicrobial peptide comprising a first amphipathic helical peptide and a second amphipathic helical peptide coupled by a linker domain forming a helix-turn-helix scaffold formation wherein said helix-turn-helix scaffold formation has:
increased bactericidal effects compared to a single endogenous amphipathic helical peptide;
increased efficiency of attachment and/or insertion into a bacterial membrane compared to a single endogenous amphipathic helical peptide;
lower susceptibility to bacterial resistance compared to a single endogenous amphipathic helical peptide; and
no toxicity to mammalian cells; and
no phytotoxcicity to plant cells.

176. The antimicrobial peptide of claim 175 for use as a therapeutic agent for plants infected with and/or at risk of being infected by a bacterial pathogen.

177. The antimicrobial peptide of claim 176 for use as a therapeutic agent for plants infected with and/or at risk of being infected by gram-negative pathogens.

178. The composition of 177 for use as a topical application for plants infected with and/or at risk of being infected by CLas.

179. The composition of 178 for use as a therapeutic agent for the treatment and/or prevention of Huanglongbing (HLB).

180. An antimicrobial peptide comprising a first amphipathic helical peptide and a second amphipathic helical peptide coupled by a linker domain forming a helix-turn-helix scaffold formation.

181. The antimicrobial peptide of claim 180 wherein said first amphipathic helical peptide and said second amphipathic helical peptide are both endogenous amphipathic helical peptides from a citrus plant.

182. The antimicrobial peptide of claim 181 wherein said first amphipathic helical peptide and/or said second amphipathic helical peptide are each selected from the group consisting of: P11, 11P1, 12P, 12P1, 12P-2, 10P, and 27P, or any combination thereof.

183. The antimicrobial peptide of claim 181 wherein said first amphipathic helical peptide and/or said second amphipathic helical peptide are each selected from the group consisting of: SEQ ID NOs. 1-2, 13-15, 19, and 21, or any combination thereof.

184. The antimicrobial peptide of claim 183 wherein said linker domain comprises a peptide linker having at least four amino acids.

185. The antimicrobial peptide of claim 184 wherein said linker domain comprises a GPGR-turn having an amino acid sequence identified as SEQ ID NOs. 23.

186. The antimicrobial peptide of claim 181 wherein said first amphipathic helical peptide and said second amphipathic helical peptide are the same amphipathic helical peptide.

187. The antimicrobial peptide of claim 186 wherein said antimicrobial peptide is selected from the group consisting of: P26, 26P1, 26P2, 26P3, 26P4, 26P5, cysP30, 41P, 28P, 28P1, 28P1-2, 24P, and 58-P.

188. The antimicrobial peptide of claim 186 wherein said antimicrobial peptide is selected from the group consisting of: SEQ ID NOs. 3-12, 16-18, 20, and 22-23, or a variant thereof.

189. The antimicrobial peptide of claim 188 is encoded by a polynucleotide comprising a nucleic acid sequence.

190. The polynucleotide of claim 189 linked to a promoter to produce an expression vector.

191. A genetically altered plant or plant cell comprising the polynucleotide of claim 190 operably linked to a promotor, wherein said plant or plant cell produce said antimicrobial peptide.

192. The antimicrobial peptide of claim 188 for use as a therapeutic agent for plants infected with and/or at risk of being infected by a bacterial pathogen.

193. The antimicrobial peptide of claim 192 for use as a therapeutic agent for plants infected with and/or at risk of being infected by Candidatus Liberibacte asiaticus (CLas).

194. The composition of 193 for use as a topical application for plants infected with and/or at risk of being infected by CLas.

195. The composition of 194 for use as a therapeutic agent for the treatment and/or prevention of Huanglongbing (HLB).

196. The antimicrobial peptide of claim 180 wherein said first amphipathic helical peptide and a second amphipathic helical peptide coupled by a linker domain forming a helix-turn-helix scaffold formation has increased bactericidal effects compared to a single endogenous amphipathic helical peptide.

197. The antimicrobial peptide of claim 180 wherein said first amphipathic helical peptide and a second amphipathic helical peptide coupled by a linker domain forming a helix-turn-helix scaffold formation having increased efficiency of attachment and/or insertion into a bacterial membrane compared to a single endogenous amphipathic helical peptide.

198. The antimicrobial peptide of claim 180 wherein said first amphipathic helical peptide and a second amphipathic helical peptide coupled by a linker domain forming a helix-turn-helix scaffold formation has a lower susceptibility to bacterial resistance compared to a single endogenous amphipathic helical peptide.

199. The antimicrobial peptide of claim 180 wherein said first amphipathic helical peptide and a second amphipathic helical peptide coupled by a linker domain forming a helix-turn-helix scaffold formation has a no toxicity to mammalian cells or phytotoxcicity to plant cells.

200. An antimicrobial peptide comprising two P11 amphipathic helical peptides coupled by a linker domain forming a helix-turn-helix scaffold formation identified as amino acid SEQ ID NO. 3.

201. The antimicrobial peptide of claim 200 wherein at least one hydrophobic amino acid residue from each of said P11 amphipathic helical peptides are replaced with a cysteine residue forming a disulfide bridge between said P11 amphipathic helical peptides.

202. The antimicrobial peptide of claim 201 identified as amino acid SEQ ID NO. 9.

203. The antimicrobial peptide of claim 200 and further composing a second linker domain coupling said two P11 amphipathic helical peptides forming a cyclic scaffold formation.

204. The antimicrobial peptide of claim 203 identified as amino acid SEQ ID NO. 11.

205. The antimicrobial peptide of claim 200 is encoded by a polynucleotide comprising a nucleic acid sequence.

206. The polynucleotide of claim 205 linked to a promoter to produce an expression vector.

207. A genetically altered plant or plant cell comprising the polynucleotide of claim 205 operably linked to a promotor, wherein said plant or plant cell produce said antimicrobial peptide.

208. The antimicrobial peptide of claim 200 for use as a therapeutic agent for plants infected with and/or at risk of being infected by a bacterial pathogen.

209. The antimicrobial peptide of claim 208 for use as a therapeutic agent for plants infected with and/or at risk of being infected by Candidatus Liberibacte asiaticus (CLas).

210. The composition of 209 for use as a topical application for plants infected with and/or at risk of being infected by CLas.

211. The composition of 210 for use as a therapeutic agent for the treatment and/or prevention of Huanglongbing (HLB).

212. The antimicrobial peptide of claim 200 wherein said first amphipathic helical peptide and a second amphipathic helical peptide coupled by a linker domain forming a helix-turn-helix scaffold formation has increased bactericidal effects compared to a single endogenous amphipathic helical peptide.

213. The antimicrobial peptide of claim 200 wherein said first amphipathic helical peptide and a second amphipathic helical peptide coupled by a linker domain forming a helix-turn-helix scaffold formation having increased efficiency of attachment and/or insertion into a bacterial membrane compared to a single endogenous amphipathic helical peptide.

214. The antimicrobial peptide of claim 200 wherein said first amphipathic helical peptide and a second amphipathic helical peptide coupled by a linker domain forming a helix-turn-helix scaffold formation has a lower susceptibility to bacterial resistance compared to a single endogenous amphipathic helical peptide.

215. An antimicrobial peptide comprising SEQ ID NO. 3, or SEQ ID NO. 9, or SEQ ID NO. 11, or a sequence having 75% sequence identity with any of the identified sequences.


SEQUENCE LISTING (23 sequences)

SEQ ID NO: 1 - protein, 11 aa, Citrus sinensis
LIKLIKKILKK

SEQ ID NO: 2 - protein, 11 aa, Citrus sinensis
LIRLIRRILRR

SEQ ID NO: 3 - protein, 26 aa, Artificial Sequence
  P26
LIKLIKKILKKGPGRKKLIKKILKIL

SEQ ID NO: 4 - protein, 26 aa, Artificial Sequence
  26P1
LIRLIRRILRRGPGRRRLIRRILRIL

SEQ ID NO: 5 - protein, 26 aa, Artificial Sequence
  26P2
LIRLLRRILRRGPGRRRLIRRLLRIL

SEQ ID NO: 6 - protein, 26 aa, Artificial Sequence
  26P3
LIRLLREILRRGPGRERLIRRLLRIL

SEQ ID NO: 7 - protein, 26 aa, Artificial Sequence
  26P4
LIRLILRILRRGPGRRRLIRLILRIL

SEQ ID NO: 8 - protein, 26 aa, Artificial Sequence
  26P5
LIRLISRILRRGPGRRRLIRSILRIL

SEQ ID NO: 9 - protein, 26 aa, Artificial Sequence
  cysP26
LIKLCKKILKKGPGRKKLIKKCLKIL

SEQ ID NO: 10 - protein, 30 aa, Artificial Sequence
  P30
LIKLIKKILKKGPGRKKLIKKILKILGPGR

SEQ ID NO: 11 - protein, 30 aa, Artificial Sequence
  cycP30
GRLIKLIKKILKKGPGRKKLIKKILKILGP

SEQ ID NO: 12 - protein, 41 aa, Artificial Sequence
  41P
KKLIKKILKILGPGRKKLIKEILKILGPGRKKLIKKILKIL

SEQ ID NO: 13 - protein, 12 aa, Citrus sinensis
KRIVQRIKDFLR

SEQ ID NO: 14 - protein, 12 aa, Citrus sinensis
KRLVQRLKDFLR

SEQ ID NO: 15 - protein, 12 aa, Citrus sinensis
KRLIQRKRLIQR

SEQ ID NO: 16 - protein, 28 aa, Artificial Sequence
  28P
KRIVQRIKDFLRGPGRKRIVQRIKDFLR

SEQ ID NO: 17 - protein, 28 aa, Artificial Sequence
  28P1
KRLVQRLKDFLRGPGRKRLVQRLKDFLR

SEQ ID NO: 18 - protein, 28 aa, Artificial Sequence
  28P1 - 2
KRLIQRKRLIQRGPGRKRLIQRKRLIQR

SEQ ID NO: 19 - protein, 10 aa, Citrus sinensis
LYKKLSKKLL

SEQ ID NO: 20 - protein, 24 aa, Artificial Sequence
  24P
LYKKLSKKLLGPGRLYKKLSKKLL

SEQ ID NO: 21 - protein, 27 aa, Citrus sinensis
ALYLKDFKSSKSLDVSALADLKHLKRL

SEQ ID NO: 22 - protein, 58 aa, Citrus sinensis
ALYLKDFKSSKSLDVSALADLKHLKRLGPGRALYLKDFKSSKSLDVSALADLKHLKRL

SEQ ID NO: 23 - protein, 4 aa, Artificial Sequence
  Linker
GPGR